(19) United States
(12) Patent Application Publication
(10) Pub. No.: US 2008/0194681 A1
(43) Pub. Date: Aug. 14, 2008

(54) Novel Inhibitors of Histone Deacetylase for the Treatment of Disease

(75) Inventors: James Malecha, San Diego, CA (US); Stewart Noble, San Diego, CA (US); Christian Hassig, Mira Mesa, CA (US); Paul Wash, San Diego, CA (US); Brandon Wiley, Philadelpha, PA (US); Charles Lawrence, San Diego, CA (US); Timothy Hoffman, San Diego, CA (US); Celine Bonnefous, San Diego, CA (US); NIcholas Smith, San Diego, CA (US)

Correspondence Address: GLOBAL PATENT GROUP - KAL;ATTN: MS LAVERN HALL, 10411 Clayton Road, Suite 304, St. Louis, MO, 63131, US

(73) Assignee: Kalypsys, Inc., San Diego, CA (US)

(21) Appl. No.: 11/792,624

(22) PCT Filed: Dec. 9, 2005
(86) PCT No.: PCT/US05/44743

§ 371 Date: Nov. 6, 2007

Related U.S. Application Data
(60) Provisional application No. 60/634,844, filed on Dec. 9, 2004.
(60) Provisional application No. 60/692,856, filed on Jun. 22, 2005.

(51) Int. Cl. A61K 31/265; C07C 327/32; A61P 9/00; A61P 7/06; A61P 35/00; A61P 35/04

(52) U.S. Cl. 514/513; 558/253

(57) ABSTRACT

Disclosed herein are carbonyl compounds of Formula: (I) as described herein. Compounds as modulators of his-tone deacetylase (HDAC), pharmaceutical compositions comprising the same, and methods of treating disease using the same are disclosed.

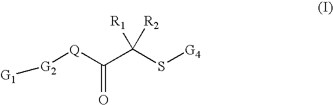

This application claims priority to U.S. Provisional applications 60/634,844 dated Dec. 9, 2004, and 60/692,856 dated Jun. 22, 2005.

### FIELD OF THE INVENTION

The present invention is directed to carbonyl compounds as inhibitors of histone deacetylase (HDAC). These compounds are useful in treating disease states including cancers, autoimmune diseases, tissue damage, central nervous system disorders, neurodegenerative disorders, fibrosis, bone disorders, polyglutamine-repeat disorders, anemias, thalassemias, inflammatory conditions, cardiovascular conditions, and disorders in which angiogenesis plays a role in pathogenesis.

### BACKGROUND OF THE INVENTION

Histone proteins organize DNA into nucleosomes, which are regular repeating structures of chromatin. The acetylation status of histones alters chromatin structure, which, in turn, is involved in gene expression. Two classes of enzymes can affect the acetylation of histones—histone acetyltransferases (HATs) and histone deacetylases (HDACs). A number of HDAC inhibitors have been characterized. However, to date no effective candidate for cancer therapy has been identified. Therefore, there is a need in the art to discover HDAC inhibitors that have effective anti-tumor activity.

### SUMMARY OF THE INVENTION

Disclosed herein are compounds of the invention having Formula I and related formulae as described herein, including their pharmaceutically acceptable salts, amides, esters, and prodrugs:

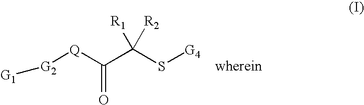

G₂ is an optionally substituted phenyl with one or more substitutents R₃;

G₁ is selected from the group consisting of W and Z;

W is independently selected from the group consisting of
i) an alkoxy of formula —(X₁)ₙ₁—O—(X₂)ₙ₂—X₃, where
   each X₁ and each X₂ is each independently selected from the group consisting of optionally substituted lower alkylene, lower alkenylene, lower alkynylene, aryl, and heteroaryl;
   X₃ is selected from the group consisting of substituted alkyl, substituted aryl, substituted heteroaryl, optionally substituted heteroalkyl, optionally substituted cycloalkyl, optionally substituted heterocycloalkyl, optionally substituted aralkyl, optionally substituted heteroaralkyl, optionally substituted fused polycyclic aryl and cycloalkyl, optionally substituted fused polycyclic aryl and heterocycloalkyl, optionally substituted linked bi-aryl, optionally substituted linked aryl-heteroaryl, optionally substituted linked heteroaryl-heteroaryl, optionally substituted linked aryl-heterocycloalkyl, an amine of the formula —NX₄X₅, an alkoxy of the the formula —OX₄, and a thioether of the formula —SX₄, where each X₄ and each X₅ are each independently hydrogen, optionally substituted lower alkyl, optionally substituted aryl, optionally substituted heteroaryl, optionally substituted aralkyl, and optionally substituted heteroaralkyl;
   or X₄ and X₅ taken together form an optionally substituted heterocycloalkyl which is optionally fused with optionally substituted aryl or heteroaryl; and
   n₁ and n₂ are each independently 0, 1, 2 or 3;
ii) an amide of formula —(X₆)ₙ₃—C(O)—N((X₇)ₙ₄—X₈)X₉ or —(X₄)ₙ₃—N(X₉)C(O)—(X₇)ₙ₄—X₈, where
   each X₆ and each X₇ is independently selected from the group consisting of optionally substituted lower alkylene, lower alkenylene, lower alkynylene, aryl, and heteroaryl;
   X₈ is selected from the group consisting of substituted lower alkyl, optionally substituted aryl, substituted heteroaryl, substituted heteroalkyl, optionally substituted heterocycloalkyl, optionally substituted aralkyl, optionally substituted heteroaralkyl, optionally substituted fused polycyclic aryl and cycloalkyl, optionally substituted fused polycyclic aryl and heterocycloalkyl, optionally substituted linked bi-aryl, optionally substituted linked aryl-heteroaryl, optionally substituted linked heteroaryl-heteroaryl, optionally substituted linked aryl-heterocycloalkyl, an amine of the formula —NX₁₀X₁₁, an alkoxy of the the formula —OX₁₀, and a thioether of the formula —SX₁₀, where X₁₀ and X₁₁ are each independently hydrogen, optionally substituted lower alkyl, optionally substituted aryl, optionally substituted heteroaryl, optionally substituted aralkyl, and optionally substituted heteroaralkyl, provided that X₁₀ is neither H nor unsubstituted alkyl; or, if X₉ is not H, X₈ may additionally be selected from the group consisting of lower alkyl, aryl, heteroaryl, and heteroalkyl;
   X₉ is selected from the group consisting of hydrogen, optionally substituted lower alkyl, optionally substituted heteroalkyl, optionally substituted aryl, optionally substituted heteroaryl, optionally substituted heterocycloalkyl, hydroxyl, and optionally substituted alkoxy;
   or X₈ and X₉ taken together form an optionally substituted heterocycloalkyl; and
   each n₃ and n₄ is independently 0, 1, 2, or 3;
iii) an amino of formula —(X₁₂)ₙ₅—N((X₁₃)ₙ₆—X₁₄)X₁₅, where
   X₁₂ and X₁₃ is each independently selected from the group consisting of lower alkylene, lower alkenylene, lower alkynylene, aryl, and heteroaryl;
   X₁₄ is selected from the group consisting of substituted lower alkyl, optionally substituted aryl, substituted heteroaryl, substituted heteroalkyl, optionally substituted heterocycloalkyl, optionally substituted aralkyl, optionally substituted heteroaralkyl, optionally substituted fused polycyclic aryl and cycloalkyl, optionally substituted fused polycyclic aryl and heterocycloalkyl, optionally substituted linked bi-aryl, optionally substituted linked aryl-heteroaryl, optionally substituted linked heteroaryl-heteroaryl, optionally substituted linked aryl-heterocycloalkyl, an amine of the formula —NX₁₆X₁₇, an alkoxy of the the formula —OX₁₆, and a thioether of the formula —SX₁₆, where X₁₆ and X₁₇ are each independently hydrogen, optionally substituted lower alkyl, optionally substituted aryl, optionally substituted heteroaryl, optionally substituted aralkyl, and optionally substituted heteroaralkyl;
   X₁₅ is selected from the group consisting of hydrogen, optionally substituted lower alkyl, optionally substituted heteroalkyl, optionally substituted aryl, optionally substituted heteroaryl, optionally substituted heterocycloalkyl, hydroxyl, and optionally substituted alkoxy;
   or n₆ is 0 and X₁₄ and X₁₅, taken together with the nitrogen to which they are attached, form a substituted five-membered or six-membered heteroaromatic or heterocyclic ring;
   with the proviso that when X₁₄ and X₁₅ are taken together with the nitrogen to which they are attached to form a six-membered heterocyclic ring that contains an endocyclic oxygen, then it is not N-morpholino; and
   n₅ and n₆ are each independently 0, 1, 2, or 3;
iv) a thioether or thiol of formula —(X₁₈)ₙ₇—S—(X₁₉)ₙ₈—X₂₀, or the higher oxide forms —(X₁₈)ₙ₇—S(O)—(X₁₉)ₙ₈—X₂₀ and —(X₁₈)ₙ₇—SO₂—(X₁₉)ₙ₈—X₂₀, wherein
   X₁₈ and X₁₉ are each independently selected from the group consisting of lower alkylene, lower alkenylene, lower alkynylene, aryl, and heteroaryl;
   X₂₀ is selected from the group consisting of substituted lower alkyl, substituted aryl, substituted heteroaryl, optionally substituted heteroalkyl, optionally substituted heterocycloalkyl, optionally substituted aralkyl, optionally substituted heteroaralkyl, optionally substituted fused polycyclic aryl and cycloalkyl, optionally substituted fused polycyclic aryl and heterocycloalkyl, optionally substituted linked bi-aryl, optionally substituted linked aryl-heteroaryl, optionally substituted linked heteroaryl-heteroaryl, optionally substituted linked aryl-heterocycloalkyl, an alkoxy of the the formula —OX₂₁, and a thioether of the formula —SX₂₁, where X₂₁ and X₂₂ are each independently hydrogen, optionally substituted lower alkyl, optionally substituted aryl, optionally substituted heteroaryl, optionally substituted aralkyl, and optionally substituted heteroaralkyl; and
   each n₇ and n₈ is independently 0, 1, 2, or 3; further wherein if n₉ is 0 and G₁=W, then X₂₀ excludes perfluoroalkyl; further wherein, if G=Z and R₂₀=unsubstituted alkyl or unsubstituted aralkyl then X₂₀ excludes perfluoroalkyl;
v) a moiety of the structure —(X₂₃)ₙ₉—N(X₂₅)C(O)—V—(X₂₄)ₙ₁₀—X₂₆ or —(X₂₃)ₙ₉—V—C(O)N((X₂₄)ₙ₁₀—X₂₅)X₂₆, wherein
   V is independently selected from O and S;
   X₂₃ and X₂₄ are each independently selected from the group consisting of lower alkylene, lower alkenylene, lower alkynylene, aryl, and heteroaryl;
   X₂₅ and X₂₆ are each independently selected from the group consisting of hydrogen, optionally substituted lower alkyl, optionally substituted heteroalkyl, optionally substituted aryl, optionally substituted heteroaryl, optionally substituted heterocycloalkyl, optionally substituted aralkyl, optionally substituted heteroaralkyl, optionally substituted fused polycyclic aryl and cycloalkyl, optionally substituted fused polycyclic aryl and heterocycloalkyl, optionally substituted linked bi-aryl, optionally substituted linked aryl-heteroaryl, optionally substituted linked heteroaryl-heteroaryl, optionally substituted linked aryl-heterocycloalkyl, an amine of the formula —NX₂₇X₂₈, an alkoxy of the the formula —OX₂₇, and a thioether of the formula —SX₂₇, where each X₂₇ and each X₂₈ are each independently hydrogen, optionally substituted lower alkyl, optionally substituted aryl, optionally substituted heteroaryl, optionally substituted aralkyl, and optionally substituted heteroaralkyl;
   or n₁₀ is 0 and X₂₅ and X₂₆, taken together with the nitrogen to which they are attached, form an optionally substituted five-membered or six-membered heteroaromatic or heteroaliphatic ring; and
   n₉ and n₁₀ are each independently 0, 1, 2, or 3; and
vi) a moiety of the structure —X₂₉—X₃₀, wherein
   each X₂₉ is independently selected from the group consisting of optionally substituted C₁-C₁₀ alkylene, optionally substituted C₁-C₁₀ alkenylene, and optionally substituted C₁-C₁₀ alkynylene;
   each X₃₀ is independently selected from the group consisting of optionally substituted aryl, optionally substituted heteroaryl, optionally substituted heterocycloalkyl, optionally substituted fused polycyclic aryl and cycloalkyl, optionally substituted fused polycyclic aryl and heterocycloalkyl, optionally substituted linked bi-aryl, optionally substituted linked aryl-heteroaryl, optionally substituted linked heteroaryl-heteroaryl, optionally substituted linked aryl-heterocycloalkyl; further wherein if G₁=Z. then X₃₀ is substituted.

Z is selected from the group consisting of
i) an N-sulfonamido of structure

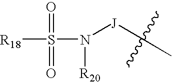

wherein R₁₈ is selected from the group consisting of —(X₃₁)ₙ₁₁—X₃₂, and —NX₃₅X₃₆—, wherein each X₃₁ is independently selected from the group consisting of optionally substituted lower alkylene, lower alkenylene, and lower alkynylene;
X₃₅ is selected from the group consisting of hydrogen, optionally substituted lower alkyl, and optionally substituted lower heteroalkyl;
X₃₆ is selected from the group consisting of optionally substituted aryl, optionally substituted heteroaryl, and optionally substituted fused polycyclic aryl and cycloalkyl;
J is —(CH₂)ₖ—, wherein k is 0-3;
each n₁₁ is independently 1, 2, or 3;
each X₃₂ is independently selected from the group consisting of substituted and monocyclic aryl, heteroaryl, cycloalkyl, and heterocycloalkyl;
and wherein R₂₀ is hydrogen, optionally substituted lower alkyl, optionally substituted lower aralkyl, optionally substituted aryl, optionally substituted heteroalkyl, and optionally substituted heteroaralkyl; and
ii) an S-sulfonamido of formula

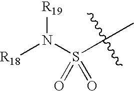

wherein R₁₈ is independently selected from the group consisting of —(X₃₃)ₙ₁₂—X₃₄, wherein X₃₃ is independently selected from the group consisting of optionally substituted lower alkylene, lower alkenylene, and lower alkynylene;
n₁₂ is 1,2, or 3; and
X₃₄ is an optionally substituted monocyclic phenyl, where the substitutents cannot be taken in together to form a ring fused with the phenyl moiety;
wherein R₁₉ is hydrogen, optionally substituted lower alkyl, optionally substituted lower aralkyl, optionally substituted aryl; optionally substituted heteroalkyl, and optionally substituted heteroaralkyl;
or R₁₈ taken together with Rig and the nitrogen to which they are attached forms an optionally substituted heterocycloalkyl;
R₁ and R₂ are each independently selected from the group consisting of hydrogen, optionally substituted lower alkyl, and halogen, or taken together form optionally substituted cycloalkyl;
R₃ is selected from the group consisting of hydrogen, optionally substituted lower alkyl, optionally substituted aryl, optionally substituted heteroaryl, optionally substituted heteroalkyl, halogen, optionally substituted amino, and hydroxyl;
Q is selected from the group consisting of a bond, —(CH₂)ₘ—, —CH₂)ₘNR₂₁—, —(CH₂)ₘ(CO)—, —(CH₂)ₘNR₂₁(CO)—, —(CH₂)ₘNR₂₁(CO)— and —(CH₂)ₘC(O)NR₂₁—, wherein m is 0-7, optionally substituted lower alkylene, optionally substituted lower alkynylene, optionally substituted lower heteroalkyl, and optionally substituted lower heteroalkynylene, wherein if Q is not symmetric, Q may be attached in either order;
R₂₁ is selected from the group of hydrogen, alkenyl, and alkyl, wherein alkyl is C₁ to C₈; and G₄ is selected from the group consisting of acyl, aryl, alkyl, heteroaryl, and Z, wherein Z has the structural Formula (II)

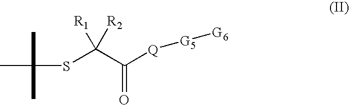

G₅ is selected from the group consisting of monocyclic aryl, polycyclic aryl, monocyclic heteroaryl, and polycyclic heteroaryl; G₄ is selected from the group consisting of optionally substituted alkylthio and optionally substituted arylthio to form a disulfide with the alkylthio or arylthio substituents;

With the proviso that the said compound is not the following:

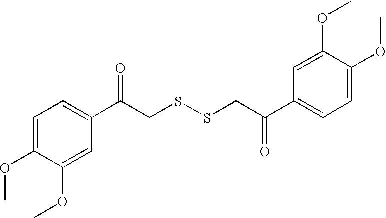

The invention also provides pharmaceutical compositions comprising a compound having structural formula I or an ester, slat, amide, or prodrug thereof, which are capable of inhibiting the catalytic activity of histone deacetylase (HDAC).

The invention also provides methods and compositions for treating diseases in mammals using compounds of the invention, including but not limited to, treating cancers, autoimmune diseases, tissue damage, central nervous system disorders, neurodegenerative disorders, fibrosis, bone disorders, polyglutamine-repeat disorders, anemias, thalassemias, inflammatory conditions, cardiovascular conditions, and disorders in which angiogenesis plays a role in pathogenesis. The invention further provides methods of inhibiting the catalytic activity and cellular function of histone deacetylase (HDAC).

### DETAILED DESCRIPTION OF THE PREFERRED EMBODIMENT

As used in the present specification, the following terms have the meanings indicated.

The term “pharmaceutically acceptable salt” refers to a formulation of a compound that does not cause significant irritation to an organism to which it is administered and does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, methanesulfonic acid, ethanesulfonic acid, prtoluenesulfonic acid, salicylic acid and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like.

The terms “physiologically acceptable” and “physiologically compatible” refers to excipients, products, or hydrolysis products of disclosed molecular embodiments of the invention. By way of example, protected thiol prodrug embodiments may release acids upon hydrolysis of the protected thiol. Physiologically acceptable excipients and acids are those that do not abrogate the biological activity or properties of the compound, and are nontoxic. “Physiologically acceptable” and “pharmaceutically acceptable” may be coextensive terms.

The term “ester” refers to a chemical moiety with formula —(R)ₙ—COOR′, where R and R′ are independently selected from the group consisting of alkyl, cycloalkyl, aryl, heteroaryl (bonded through a ring carbon) and heteroalicyclic (bonded through a ring carbon), and where n is 0 or 1. Any amine, hydroxy, or carboxyl side chain on the compounds of the present invention can be esterified. The procedures and specific groups to be used to achieve makes such esters are known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference in its entirety.

An “amide” is a chemical moiety with formula —(R)ₙ—C(O)NHR′ or —(R)ₙ—NHC(O)R′, where R and R′ are independently selected from the group consisting of alkyl, cycloalkyl, aryl, heteroaryl (bonded through a ring carbon) and heteroalicyclic (bonded through a ring carbon), and where n is 0 or 1. An amide may be an amino acid or a peptide molecule attached to a molecule of the present invention, thereby forming a prodrug.

Any amine, hydroxy, or carboxyl side chain on the compounds of the present invention can be esterified or amidified. The procedures and specific groups to be used to achieve this end is known to those of skill in the art and can readily be found in reference sources such as Greene and Wuts, Protective Groups in Organic Synthesis, 3^{rd} Ed., John Wiley & Sons, New York, N.Y., 1999, which is incorporated herein in its entirety.

A “prodrug” refers to an agent that is converted into the parent drug in vivo. Prodrugs are often useful because, in some situations, they may be easier to administer than the parent drug. They may, for instance, be bioavailable by oral administration whereas the parent is not; The prodrug may also have improved solubility over the parent drug. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. Yet another example of a prodrug are protected thiol compounds. Thiols bearing hydrolyzable protecting groups can unmask protected SH groups prior to or simultaneous to use. As shown below, the moiety —C(O)—R_{E} of a thioester may be hydrolyzed to yield a thiol and a pharmaceutically acceptable acid HO—C(O)—R_{E}.

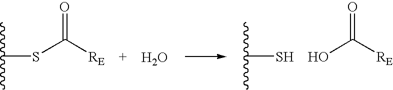

The term “thiol protecting group” refers to thiols bearing hydrolyzable protecting groups that can unmask protected SH groups prior to or simultaneous to use. Preferred thiol protecting groups include but are not limited to thiol esters which release pharmaceutically acceptable acids along with an active thiol moiety. Such pharmaceutically acceptable acids are generally nontoxic and do not abbrogate the biological activity of the active thiol moiety. Examples of pharmaceutically acceptable acids include, but are not limited to: N,N-diethylglycine; 4-ethylpiperazinoacetic acid; ethyl 2-methoxy-2-phenylacetic acid; N,N-dimethylglycine; (nitrophenoxysulfonyl)benzoic acid; acetic acid; maleic acid; fumaric acid; benzoic acid; tartraric acid; natural amino acids (like glutamate, aspartate, cyclic amino acids such proline); D-amino acids; butyric acid; fatty acids like palmitic acid, stearic acid, oleate; pipecolic acid; phosphonic acid; phosphoric acid; pivalate (trimethylacetic acid); succinic acid; cinnamic acid; anthranilic acid; salicylic acid; lactic acid; and pyruvic acids.

The term “alkenylene” refers to a difunctional branched or unbranched hydrocarbon chain containing at least one carbon-carbon double bond. “Lower alkenylene” refers to an alkenylene group of 2 to 6 carbon atoms, containing one carbon-carbon double bond.

As used herein, the term “alkyl” refers to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 4 carbon atoms. The alkyl group of the compounds of the invention may be designated as “C₁-C₅ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl.

The alkyl group may be substituted or unsubstituted. When substituted, any group(s) besides hydrogen can be the substitutent group(s). When substituted, the substituent group(s) is(are) one or more group(s) individually and independently selected from the following non-limiting illustrative list: alkyl, cycloalkyl, aryl, heteroaryl, heterocycloalkyl, hydroxy, alkoxy, aryloxy, O, S, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups, and the protected derivatives thereof. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. Each substituent group may be further substituted.

The term “alkylene,” as used herein, alone or in combination, refers to a saturated aliphatic group derived from a straight or branched chain saturated hydrocarbon attached at two or more positions, such as methylene (—CH₂—).

The term “alkynylene” refers to a difunctional branched or unbranched hydrocarbon chain containing at least one carbon-carbon triple bond. “Lower alkynylene” refers to an alkynylene group of 2 to 6 carbon atoms, containing one carbon-carbon triple bond.

The term “aralkyl” as used herein refer to an alkyl radical as defined above in which at least one hydrogen atom is replaced by an aryl radical as defined below.

Unless otherwise indicated, when a group is described as “optionally substituted,” it is meant that the group may be substituted with one or more substituents selected from the following non-limiting illustrative list: hydroxy, alkyl,-alkoxy, aryloxy, cycloalkyl, aryl, carbocyclic cycloalkyl, carbocyclic aryl, heteroaryl, heterocycloalkyl, O, S, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups, and protected derivatives thereof. Protecting groups that may form the protective derivatives of the substituents recited above are known to those of skill in the art and may be found in references such as Greene and Wuts, above. Each optional substituent may be further optionally substituted. Optionally substituted groups may be unsubstituted.

The term “halo” or, alternatively, “halogen” means fluoro, chloro, bromo or iodo. Preferred halo groups are fluoro, chloro and bromo. The terms “haloalkyl,” “haloalkenyl,” “haloalkynyl” and “haloalkoxy” include alkyl, alkenyl, alkynyl and alkoxy structures, that are substituted with one or more halo groups or with combinations thereof. The terms “fluoroalkyl” and “fluoroalkoxy” include haloalkyl and haloalkoxy groups, respectively, in which the halo is fluorine.

The term “hetero” in such terms as “heteroalkyl,” “heteroalkenyl,” “heteroalkynyl,” “heterocycloalkyl,” and “heteroaryl” refers to groups in which one or more of the backbone atoms is selected from an atom other than carbon, e.g., oxygen, nitrogen, sulfur, phosphorus or combinations thereof.

Cyclic alkyl moeities contain one or more covalently closed ring structures. Cyclic alkyl moeities can have a single ring (monocyclic) or two or more rings (polycyclic or multicyclic). Polycyclic groups include fused polycyclic groups wherein rings share adjacent pairs of backbone atoms, and linked cyclic groups wherein the rings are separate but linked: In fused polycyclic groups, rings may share adjacent carbon atoms, or may share non-carbon atoms such as N. Linked polycyclic groups may be connected by a bond or a linker. Polycyclic groups can be linked by an optionally substituted alkyl moiety including but not limited to saturated alkyl linkers, or unsaturated alkyl linkers such as alkylene (e.g., methylene, ethylene, or propylene) or alkynylene linkers.

The term “carbocyclic” refers to a compound which contains one or more covalently closed ring structures, wherein the atoms forming the backbone of the ring are all carbon atoms.

The term “heterocyclic” refers to a compound with contains one or more covalently closed ring structures, wherein at least one ring backbone contains at least one atom which is different from carbon. Generally, heterocyclic groups can contain one to four heteroatoms, each selected from O, S and N, wherein each ring has from 4 to 10 atoms in the ring. Generally, heterocyclic rings do not contain two adjacent O or S atoms. An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl.

The term “cycloalkyl” refers to an aliphatic cyclic alkyl moiety wherein the ring is either completely saturated, partially unsaturated, or fully unsaturated, wherein if there is unsaturation, the conjugation of the pi-electrons in the ring do not give rise to aromaticity. The term “cycloalkyl” may refer to a monocyclic or polycyclic group. Cycloalkyl groups may be fused or linked to other cyclic alkyl moeities. A cycloalkyl group may be optionally substituted. Preferred cycloalkyl groups include groups having from three to twelve ring atoms, more preferably from 5 to 10 ring atoms. The term “carbocyclic cycloalkyl” refers to a monocyclic or polycyclic cycloalkyl group which contains only carbon and hydrogen. The term “heterocycloalkyl” refers to a monocyclic or polycyclic cycloalkyl group wherein at least one ring backbone contains at least one atom which is different from carbon.

A heterocycloalkyl group refers to a cycloalkyl group that includes at least one heteroatom selected from nitrogen, oxygen and sulfur. Heterocycloalkyl groups may be fused with one or more aryl, heteroaryl, cycloalkyl, or heterocycloalkyl groups. Heterocycloalkyl groups may be linked with one or more aryl, heteroaryl, cycloalkyl, or heterocycloalkyl groups. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycloalkyl (non-aromatic heterocyclic groups) are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl.

The terms “aryl” or “aromatic” refer to a group which has at least one ring having a conjugated pi electron system. Aryl groups can be carbocylic aryl groups or heteroaryl groups. The term “carbocyclic aryl” refers to a group (e.g., phenyl) in which all ring backbone atoms are carbon. The terms “heteroaryl” or “heteroaromatic” refer to an aryl (aromatic) group that includes one or more ring heteroatoms selected from nitrogen, oxygen and sulfur. Aryl groups may be optionally substituted. Aryl groups may be monocyclic or polycyclic. Polycyclic aryl groups may be fused or linked. Polycyclic aryl groups can be fused or linked to aryl groups or cycloalkyl groups.

The term “heteroaryl,” as used herein, alone or in combination, refers to 3 to 7 membered, preferably 5 to 7 membered, unsaturated heterocyclic rings wherein at least one atom is selected from the group consisting of O, S, and N. Heteroaryl groups are exemplified by: unsaturated 3 to 7 membered heteromonocyclic groups containing 1 to 4 nitrogen atoms, for example, pyridinyl, imidazolyl, pyrimidinyl, pyrazolyl, triazolyl, pyrazinyl, tetrazolyl, furyl, thienyl, isoxazolyl thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Polycyclic heteroaryl groups may be attached through carbon ring backbone atoms, or may be attached through ring backbone heteroatoms, especially N, depending on structure of the group. For instance, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). Polycyclic heteroaryl groups include benzo-fused ring systems and ring systems substituted with one or two oxo (═O) moieties such as pyrrolidin-2-one. The term also embraces radicals where heterocyclic radicals are fused with aryl radicals. Examples of such fused polycyclic radicals include benzofuryl, benzothienyl, methylenedioxyphenyl, ethylenedioxyphenyl, and the like.

An “O-carboxy” group refers to a RC(═O)O— group, where R is as defined herein.

A “C-carboxy” group refers to a —C(═O)OR groups where R is as defined herein.

An “acyl” group refers to a —C(═O)R group.

An “acetyl” group refers to a —C(═O)CH₃, group.

A “trihalomethanesulfonyl” group refers to a X₃CS(═O)₂— group where X is a halogen.

A “cyano” group refers to a —CN group.

An “isocyanato” group refers to a —NCO group,

A “thiocyanato” group refers to a —CNS group.

An “isothiocyanato” group refers to a —NCS group.

A “sulfinyl” group refers to a —S(═O)—R group, with R as defined herein.

A “S-sulfonamido” group refers to a —S(═O)₂NR, group, with R as defined herein.

A “N-sulfonamido” group refers to a RS(═O)₂NH— group with R as defined herein.

A “trihalomethanesulfonamido” group refers to a X₃CS(═O)₂NR— group with X and R as defined herein.

An “O-carbamyl” group refers to a —OC(═O)—NR, group-with R as defined herein.

An “N-carbamyl” group refers to a ROC(═O)NH— group, with R as defined herein.

An “O-thiocarbamyl” group refers to a —OC(═S)—NR, group with R as defined herein.

An “N-thiocarbamyl” group refers to an ROC(═S)NH— group, with R as defined herein.

A “C-amido” group refers to a —C(═O)—NR₂ group with R as defined herein.

An “N-amido” group refers to a RC(═O)NH— group, with R as defined herein.

The term partially halogenated alkyl refers to an alkyl group having both hydrogen and halogen substituents.

The term “perhaloalkyl” refers to an alkyl group where all of the hydrogen atoms are replaced by halogen atoms.

The term “lower perfluoroalkoxy” refers to a radical —O—(CX₂)ₙCX₃ where X is any halogen, preferable F or Cl, and n is 1-5.

When two substituents taken together along with the two ring carbons to which they are attached, form a ring, it is meant that the following structure:

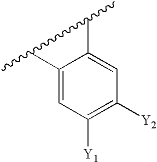

is, for example, representative of a structure such as the following:

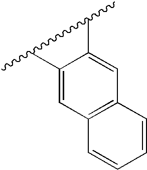

In the above example, Y₁ and Y₂, taken together along with the two ring carbons to which they are attached, form a six-membered aromatic ring.

The term “optionally substituted” means the anteceding group may be substituted or unsubstituted. When substituted, the substituents of an “optionally substituted” group may include, without limitation, one or more substituents independently selected from the following groups or designated subsets thereof, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, arylthio, lower alkylsulfinyl, lower alkylsulfonyl, arylsulfinyl, arylsulfonyl, arylthio, sulfonate, sulfonic acid, trisubstituted silyl, N₃, NHCH₃, N(CH₃)₂, SH, SCH₃, C(O)CH₃, CO₂CH₃, CO₂H, C(O)NH₂, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH2CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended.

The term R or the term R′, appearing by itself and without a number designation, unless otherwise defined, refers to an optionally substituted moiety selected from the group consisting of alkyl, cycloalkyl, heteroalkyl, aryl, heteroaryl and heterocycloalkyl. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence.

The term “bond” refers to a covalent linkage between two atoms, or two moieties when the atoms joined by the bond are considered to be part of larger substructure. A bond may be single, double, or triple unless otherwise specified.

Solubility is a thermodynamic parameter and plays an important role in the determination of a drug's bioavailability. Since a drug must be soluble in the gastrointestinal fluid to be orally active, the rate and extent of dissolution depend critically upon intrinsic water solubility (neutral species solubility) (Dressman, J.; Amindo, G. L.,; Reppas, C.; Shah. V. P. Pharm. Res., 1998, 15, 11.) Experimental and computational approaches to estimate solubility and permeability in drug discovery and development setting have been described (Lipinski C. A. et al. 1997 Adv. Drug Deliv. Rev. 23, 3-25) Adv. Drug Deliv. Rev. 23, 3-25 ). Traditional analytical methods define solubility as the concentration of material in solution at equilibrium with its solid form. In this method a compound is extensively shaken in the buffer of choice, filtered through a microppre membrane, and the concentration of dissolved compound in the filtrate determined. This approach results in a thermodynamic solubility assessment. For discovery, it is beneficial to measure kinetic solubility in which a compound DMSO solution is added to aqueous buffer. Several high throughput approaches for solubility have been described, e.g. turbidimetric method (Bevan, C. and Lloyd, R. S. Anal. Chem. 2000 72, 1781-1787), nephelometric method (Avdeef, A. (2001) High throughput measurements of solubility profiles. In Pharmacokinetic Optimization in Drug Research; Biological, Physicochemical, And Computational Strategies (Testa, B. et al., eds), pp. 305-326, Verlag Helvitica Chimica Acta and). Measurement of solubility at multiple pH levels (pH 1-8), is more useful that a single pH, since many drug candidates contain ionizable groups. A solubility-pH profile provides the pH gradient of the gastrointestinal tract.

Accurate understanding of a compound's solubility is also necessary to not only prepare and dispense formulations, but also to evaluate new chemical series and provide feedback to drive synthetic optimization. Structural series of compounds are synthesized with the aim of improving solubility by the addition of various chemical moieties. Structural elements known to confer aqueous solubility on otherwise insoluble molecular entities include but are not limited to N-piperazinylethyl, N-morpholinylethyl, 1,3-dihydroxy-2N -propanoyl moieties. Common solubilizing groups often incorporated in synthetic approaches to improve solubility of molecules include amine functionality, such as dimethylamino, diethylamino, piperazinyl, N-methyl-N-isopropylamino, morpholino, pyrrolidino moieties, or groups bearing aliphatic alcohol functionality, such as that found in ethanolamine or glycerol.

In certain embodiments of the invention, a structural element known to confer aqueous solubility is incorporated in a compound of the invention. Such structural elements are preferably attached to synthetically accessible regions of the compound. In certain embodiments, such structural elements are attached to or incorporate synthetically available N atoms in amine or amide or sulfonamide moieties of the compound. In certain embodiments a solubilizing group is attached to or incorporates a N atom and is chosen from the group consisting of dimethylamino, diethylamino, piperazinyl, N-methyl-N-isopropylamino, morpholino, pyrrolidino moieties, or groups bearing aliphatic alcohol functionality, such as that found in ethanolamine or glycerol.

In certain embodiments, the present invention relates to a compound of Formula I where Q is a bond.

In certain embodiments of compounds of the invention, G₁ is W and W is the alkoxy of formula —(X₁)ₙ₁—O—(X₂)ₙ₂—X₃.

In certain embodiments of compounds of the invention, G₁ is W and W is the alkoxy of formula —(X₁)ₙ₁—O—(X₂)ₙ₂—X₃, and X₃ is an amine of the formula —NX₄X₅, where X₄ and X₅ are each independently hydrogen, optionally substituted lower alkyl, optionally substituted aryl, optionally substituted heteroaryl, optionally substituted aralkyl, and optionally substituted heteroaralkyl.

In certain embodiments of compounds of the invention, G₁ is W and W is the amide of formula —(X₆)ₙ₃—C(O)—N((X₇)ₙ₄—X₈)X₉ or —(X₆)ₙ₃—N(X₉)C(O)—(X₇)ₙ₄—X₈.

In certain embodiments of compounds of the invention, G₁ is W and W is the amide of formula —(X₆)ₙ₃—C(O)—N((X₇)ₙ₄—X₈)X₉ or —(X₆)ₙ₃—N(X₉)C(O)—(X₇)ₙ₄—X₈, and X₈ is optionally substituted phenyl, and X₉ is selected from the group consisting of hydrogen, optionally substituted lower alkyl and optionally substituted heteroalkyl.

In certain embodiments of compounds of the invention, G₁ is W and W is the amino of formula —(X₁₂)ₙ₅—N((X₁₃)ₙ₆—X₁₄)X₁₅.

In certain embodiments of compounds of the invention, G₁ is W and W is the amino of formula —(X₁₂)ₙ₅—N((X₁₃)ₙ₆—X₁₄)X₁₅, and X₁₄ is selected from the group consisting of substituted lower alkyl, substituted aryl, substituted heteroaryl, substituted heteroalkyl, optionally substituted heterocycloalkyl, optionally substituted aralkyl, optionally substituted heteroaralkyl, optionally substituted fused polycyclic aryl and heterocycloalkyl, optionally substituted linked bi-aryl, optionally substituted linked aryl-heteroaryl, optionally substituted linked heteroaryl-heteroaryl, optionally substituted linked aryl-heterocycloalkyl, and X₁₅ is selected from the group consisting of hydrogen and optionally substituted lower alkyl.

In certain embodiments of the compounds of the invention, G₁ is W and W is the thioether or thiol of formula —(X₁₈)ₙ₇—S—(X₁₉)ₙ₈—X₂₀.

In certain embodiments of the compounds of the invention, G₁ is W and W is the thioether or thiol of formula —(X₁₈)ₙ₇—S—(X₁₉)ₙ₈—X₂₀ and X₁₈ and X₁₉ is alkylene and n7 is 1.

In certain embodiments of the compounds of the invention, G₁ is Z- and Z is the N-sulfonamido.

In certain embodiments of the compounds of the invention, G₁ is Z- and Z is the N-sulfonamido of structure

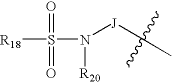

wherein R₁₈ is —NX₃₅X₃₆—.

In further embodiments, R₂₀ and R₂₁ are joined to form an optionally substituted heterocycle.

In accordance with one aspect, the present invention provides compounds of Formula I, where each compound is capable of inhibiting the catalytic activity of histone deacetylase (HDAC). In another aspect, the present invention provides pharmaceutical compositions comprising compounds of Formula I, capable of inhibiting the catalytic activity of histone deacetylase (HDAC).

In accordance with yet another aspect of the invention, the present invention provides methods and compositions for treating certain diseases or disease states.

In another aspect are compounds or compositions comprising compounds capable of inhibiting the catalytic activity of histone deacetylase (HDAC).

In some aspects of the invention, the disease to be treated by the methods of the present invention may be a hyper-proliferative condition. In some embodiments, but without limitation, the hyper-proliferative condition is selected from cancer of oral cavity and pharynx, cancer of the digestive system, cancer of the respiratory system, cancer of bones and joints, cancer of soft tissue, skin cancer, breast cancer, cancer of the genital system, cancer of the urinary system, cancer of eye and orbit, cancer of brain and other nervous system, cancer of the endocrine system, cancer of lymphoma, cancer of multiple myeloma and leukemia.

In some aspects of the invention, the disease to be treated by the methods of the present invention may be cancer. In some embodiments, but without limitation, the term cancer refers to and is selected from disorders such as tongue cancer, mouth cancer, pharynx cancer, other oral cavity cancer, esophagus cancer, stomach cancer, small intestine cancer, colon cancer, rectum cancer, anus cancer, anal canal cancer, anorectum cancer, liver cancer, intrahepatic bile duct cancer, gallbladder and other biliary organs cancer, pancreas cancer, other digestive organs cancer, larynx cancer, lung and bronchus cancer, other respiratory organs cancer, heart cancer, melanoma-skin cancer, basal cancer, squamous cancer, other non-epithelial skin cancer, uterine cervix cancer, uterine corpus cancer, ovary cancer, vulva cancer, vagina and other genital cancer, prostate cancer, testis cancer, penis and other genital cancer, urinary bladder cancer, kidney and renal pelvis cancer, ureter and other urinary organs cancer, thyroid cancer, other endocrine cancer, Hodgkin's disease cancer, non-Hodgkin's lymphoma cancer, acute lumphocytic leukemia, chronica lymphocytic leukemia, acute myeloid leukemia, chronic myeloid leukemia, other leukemias and myeloproliferative disorders such as polycythemia vera, myelofibrosis and essential thrombocythemia. The term “cancer” also encompasses Hodgkin's disease, non-Hodgkin's lymphomas, multiple myeloma and hematopoietic malignancies including leukemias (Chronic Lymphocytic Leukemia) and lymphomas including lymphocytic, granulocytic and monocytic.

Additional types of cancers which may be treated using the compounds and methods described herein include: adenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, head and neck cancer, hemangioblastoma, hepatocellular carcinoma, hepatoma, Kaposi's sarcoma, large cell carcinoma, cancer of the larynx, leiomyosarcoma, leukemias, liposarcoma, lymphatic system cancer, lymphomas, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, parathyroid tumors, pheochromocytoma, pinealoma, plasmacytomas, retinoblastoma, rhabdomyosarcoma, sebaceous gland carcinoma, seninoma, skin cancers, melanoma, small cell lung carcinoma, squamous cell carcinoma, sweat gland carcinoma, synovioma, thyroid cancer, uveal melanoma, and Wilm's tumor.

In some aspects of the invention, the disease to be treated by the methods of the present invention may be a neurological or polyglutamine-repeat disorder. In some embodiments, but without limitation, the polyglutamine-repeat disorder is selected from Huntington's disease, Spinocerebellar ataxia 1 (SCA 1), Machado-Joseph disease (MJD)/Spinocerebella ataxia 3 (SCA 3), Kennedy disease/Spinal and bulbar muscular atrophy (SBMA) and Dentatorubral pallidolusyian atrophy (DRPLA).

In some aspects of the invention, the disease to be treated by the methods of the present invention may be an anemias or thalassemia (such as Sickle Cell Disease (SCD). In some embodiments, but without limitation, the thalassemia is Sickle Cell Disease (SCD).

In some aspects of the invention, the disease to be treated by the methods of the present invention may be an inflammatory condition. In some embodiments, but without limitation, the inflammatory condition is selected from Rheumatoid Arthritis (RA), Inflammatory Bowel Disease (IBD), ulcerative colitis and psoriasis.

In some aspects of the invention, the disease to be treated by the methods of the present invention may be an autoimmnune disease. In some embodiments, but without limitation, the autoimmune disease is selected from Systemic Lupus Erythromatosus (SLE) and Multiple Sclerosis (MS).

In some aspects of the invention, the disease to be treated by the methods of the present invention may be a cardiovascular condition. In some embodiments, but without limitation, the cardiovascular condition is selected from cardiac hypertrophy and heart failure.

The terms “therapy” or “treating” as used herein refer to (1) reducing the rate of progress of a disease, or, in case of cancer reducing the size of the tumor; (2) inhibiting to some extent further progress of the disease, which in case of cancer may mean slowing to some extent, or preferably stopping, tumor metastasis or tumor growth; and/or, (3) relieving to some extent (or, preferably, eliminating) one or more symptoms associated with the disease. Thus, the term “therapeutically effective amount” as used herein refers to that amount of the compound being administered which will provide therapy or affect treatment.

In some aspects of the invention, the compounds of the present invention are also anti-tumor compounds and/or inhibit the growth of a tumor, i.e., they are tumor-growth-inhibiting compounds. The terms “anti-tumor” and “tumor-growth-inhibiting,” when modifying the term “compound,” and the terms “inhibiting” and “reducing”, when modifying the terms “compound” and/or “tumor,” mean that the presence of the subject compound is correlated with at least the slowing of the rate of growth of the tumor. More preferably, the terms “anti-tumor,” “tumor-growth-inhibiting,” “inhibiting,” and “reducing” refer to a correlation between the presence of the subject compound and at least the temporary cessation of tumor growth. The terms “anti-tumor,” “tumor-growth-inhibiting,” “inhibiting,” and “reducing” also refer to, a correlation between the presence of the subject compound and at least the temporary reduction in the mass of the tumor.

The term “function” refers to the cellular role of HDAC. The term “catalytic activity”, in the context of the invention, defines the rate at which HDAC deacetylates a substrate. Catalytic activity can be measured, for example, by determining the amount of a substrate converted to a product as a function of time. Deacetylation of a substrate occurs at the active-site of HDAC. The active-site is normally a cavity in which the substrate binds to HDAC and is deacetylated.

The term “substrate” as used herein refers to a molecule deacetylated by HDAC. The substrate is preferably a peptide and more preferably a protein. In some embodiments, the protein is a histone, whereas in other embodiments, the protein is not a histone.

The terms “treat” or “treating” or “therapy” as used herein refer to (1) reducing the rate of progress of a disease, or, in case of cancer reducing the size of the tumor; (2) inhibiting to some extent further progress of the disease, which in case of cancer may mean slowing to some extent, or preferably stopping, tumor metastasis or tumor growth; and/or, (3) relieving to some extent (or, preferably, eliminating) one or more symptoms associated with the disease. Thus, the term “therapeutically effective amount” as used herein refers to that amount of the compound being administered which will provide therapy or affect treatment.

The term “inhibit” refers to decreasing the cellular function of HDAC. It is understood that compounds of the present invention may inhibit the cellular function of HDAC by various direct or indirect mechanisms, in particular by direct or indirect inhibition of the catalytic activity of HDAC. The term “activates” refers to increasing the cellular function of HDAC.

The term “activates” refers to increasing the cellular function of HDAC. The term “inhibit” refers to decreasing the cellular function of HDAC. HDAC function is preferably the interaction with a natural binding partner and most preferably catalytic activity.

The term “modulates” refers to altering the function of HDAC by increasing or decreasing the probability that a complex forms between HDAC and a natural binding partner. A modulator may increase the probability that such a complex forms between HDAC and the natural binding partner, or may increase or decrease the probability that a complex forms between HDAC and the natural binding partner depending on the concentration of the compound exposed to HDAC, or may decrease the probability that a complex forms between HDAC and the natural binding partner. A modulator may activate the catalytic activity of HDAC, or may activate or inhibit the catalytic activity of HDAC depending on the concentration of the compound exposed to HDAC, or may inhibit the catalytic activity of HDAC.

The term “complex” refers to an assembly of at least two molecules bound to one another. The term “natural binding partner” refers to polypeptides that bind to HDAC in cells. A change in the interaction between HDAC and a natural binding partner can manifest itself as an increased or decreased probability that the interaction forms, or an increased or decreased concentration of HDAC/natural binding partner complex.

The term “contacting” as used herein refers to mixing a solution comprising a compound of the invention with a liquid medium bathing the cells of the methods. The solution comprising the compound may also comprise another component, such as dimethylsulfoxide (DMSO), which facilitates the uptake of the compound or compounds into the cells of the methods. The solution comprising the compound of the invention may be added to the medium bathing the cells by utilizing a delivery apparatus, such as a pipet-based device or syringe-based device.

The term “monitoring” refers to observing the effect of adding the compound to the cells of the method. The effect can be manifested in a change in cell phenotype, cell proliferation, HDAC catalytic activity, substrate protein acetylation levels, gene expression changes, or in the interaction between HDAC and a natural binding partner.

The term “effect” describes a change or an absence of a change in cell phenotype or cell proliferation. “Effect” can also describe a change or an absence of a change in the catalytic activity of HDAC. “Effect” can also describe a change or an absence of a change in an interaction between HDAC and a natural binding partner.

The term “cell phenotype” refers to the outward appearance of a cell or tissue or the function of the cell or tissue. Examples of cell phenotype are cell size (reduction or enlargement), cell proliferation (increased or decreased numbers of cells), cell differentiation (a change or absence of a change in cell shape), cell survival, apoptosis (cell death), or the utilization of a metabolic nutrient (e.g., glucose uptake), Changes or the absence of changes in cell phenotype are readily measured by techniques known in the art.

Pharmaceutical Compositions

The present invention also relates to a pharmaceutical composition comprising a compound of the invention, or a pharmaceutically acceptable salt, solvate, amide, ester, or prodrug thereof, as described herein and a pharmaceutically acceptable carrier, diluent, or excipient, or a combination thereof.

The term “pharmaceutical composition” refers to a mixture of a compound of the invention with other chemical components, such as carriers, diluents or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration, Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” refers to relatively nontoxic chemical compounds or agents. Such carriers may facilitate the incorporation of a compound into cells or tissues. For example, human serum albumin (HSA) is a commonly utilized carrier as it facilitates the uptake of many organic compounds into the cells or tissues of an organism.

The term “diluent” refers to chemical compounds that are used to dilute the compound of interest prior to delivery. Diluents can also be used to stabilize compounds because they can provide a more stable environment. Salts dissolved in buffered solutions (providing pH control) are utilized as diluents in the art. One commonly used buffered solution is phosphate buffered saline. It is a buffer found naturally in the blood system. Since buffer salts can control the pH of a solution at low concentrations, a buffered diluent rarely modifies the biological activity of a compound.

The compounds described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” 20th ed. Edited by Alfonso Gennaro, 2000.

Composition/Formulation

The pharmaceutical compositions of the present invention may be manufactured in a manner that is itself known, e.g., by means of conventional mixing, dissolving, granulating, dragee-making, levigating, emulsifying, encapsulating, entrapping or compression processes.

Pharmaceutical compositions for use in accordance with the present invention thus may be formulated in conventional manner using one or more pharmaceutically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For intravenous injections, the agents of the invention may be formulated in aqueous solutions, preferably in pharmaceutically compatible buffers such as Hanks's solution, Ringer's solution, or physiological saline buffer. For transmucosal administration, penetrants appropriate to the barrier to be permeated are used in the formulation. Such penetrants are generally known in the art. For other parenteral injections, the agents of the invention may be formulated in aqueous or nonaqueous solutions, preferably with pharmaceutically compatible buffers or excipients. Such excipients are generally known in the art.

For oral administration, the compounds can be formulated readily by combining the active compounds with pharmaceutically acceptable carriers or excipients well known in the art. Such carriers enable the compounds of the invention to be formulated as tablets, powders, pills, dragees, capsules, liquids, gels, syrups, elixirs, slurries, suspensions and the like, for oral ingestion by a patient to be treated. Pharmaceutical preparations for oral use can be obtained by mixing one or more solid excipient with one or more compound of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or-solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal or sublingual administration, the compositions may take the form of tablets, lozenges, or gels formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insulator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. The cosolvent system may be a 10% ethanol, 10% polyethylene glycol 300, 10% polyethylene glycol 40 castor oil (PEG-40 castor oil) with 70% aqueous solution. This cosolvent system dissolves hydrophobic compounds well, and itself produces low toxicity upon systemic administration. Naturally, the proportions of a cosolvent system may be varied considerably without destroying its solubility and toxicity characteristics. Furthermore, the identity of the cosolvent components may be varied: for example, other low-toxicity nonpolar surfactants may be used instead of PEG40 castor oil, the fraction size of polyethylene glycol 300 may be varied; other biocompatible polymers may replace polyethylene glycol, e.g., polyvinyl pyrrolidone; and other sugars or polysaccharides maybe included in the aqueous solution.

Alternatively, other delivery systems for hydrophobic pharmaceutical compounds may be employed. Liposomes and emulsions are well known examples of delivery vehicles or carriers for hydrophobic drugs. Certain organic solvents such as N-methylpyrrolidone also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

The disclosed compounds can be used for the manufacture of a medicament for use in the treatment of a condition mediated by HDAC activity.

Routes of Administration

Suitable routes of administration include local or systemic routes of administration including, but not limited to, topical, transdermal, oral, rectal, transmucosal, pulmonary, ophthalmic, intestinal, parenteral, intramuscular, subcutaneous, intravenous, intramedullary, intrathecal, direct intraventricular, intraperitoneal, intranasal, or intraocular delivery. In certain embodiments, compounds of the invention are administered topically, e,g in an ointment, patch, nasal spray, or eye drops/ointment. In certain embodiments, compounds of the invention are delivered by intestinal, parenteral, intramuscular, subcutaneous, intravenous, intramedullary, intrathecal, direct intraventricular, intraperitoneal, intranasal, or intraocular injections.

Alternately, one may administer the compound in a local rather than systemic manner, for example, via injection of the compound directly into an organ, often in a depot or sustained release formulation. Furthermore, one may administer the drug in a targeted drug delivery system, for example, in a liposome coated with organ-specific antibody. The liposomes will be targeted to and taken up selectively by the organ.

### EXAMPLES

The examples below are non-limiting and are merely representative of various aspects of the invention.

General Synthetic Methods for Preparing Compounds

Molecular embodiments of the present invention can be synthesized using standard synthetic techniques known to those of skill in the art. Compounds of the present invention can be synthesized using the general synthetic procedures set forth in Schemes I-IX

General Procedure for Reverse Sulfonamide:

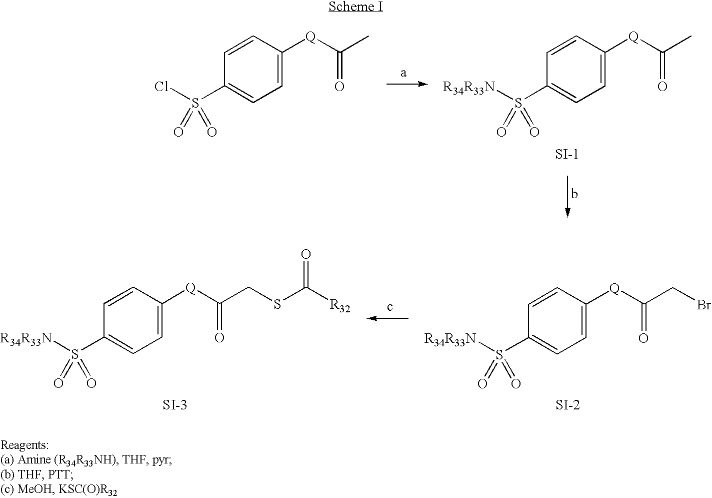

General Procedure for Sulfonamide:

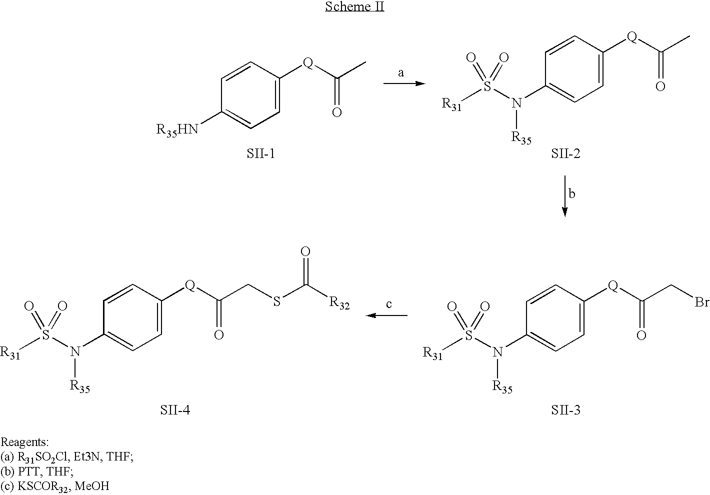

General Procedure for Ethers/Thioethers:

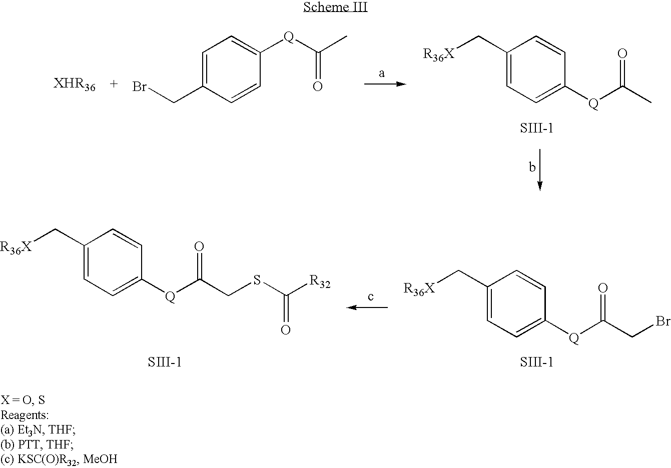

General Procedure for the Synthesis of Tertiary Amines (a):

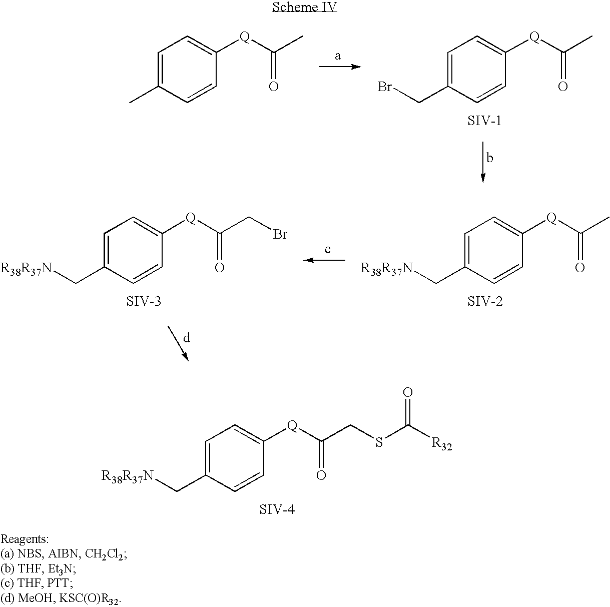

General Procedure for the Synthesis of Ureas:

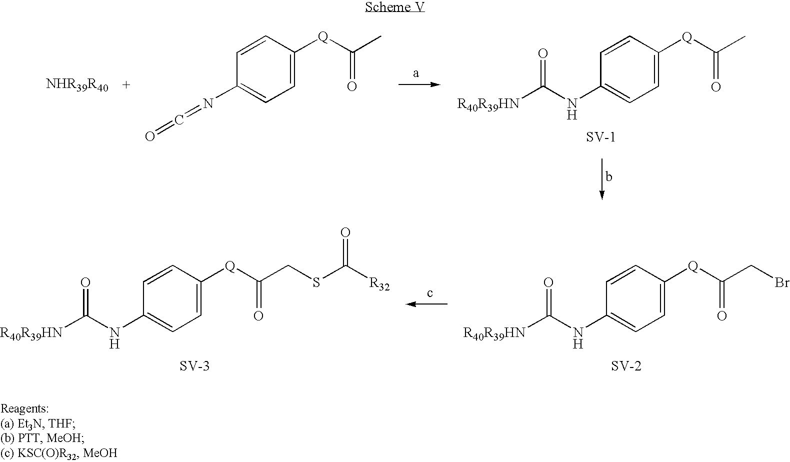

General Procedure for the Synthesis of Amides:

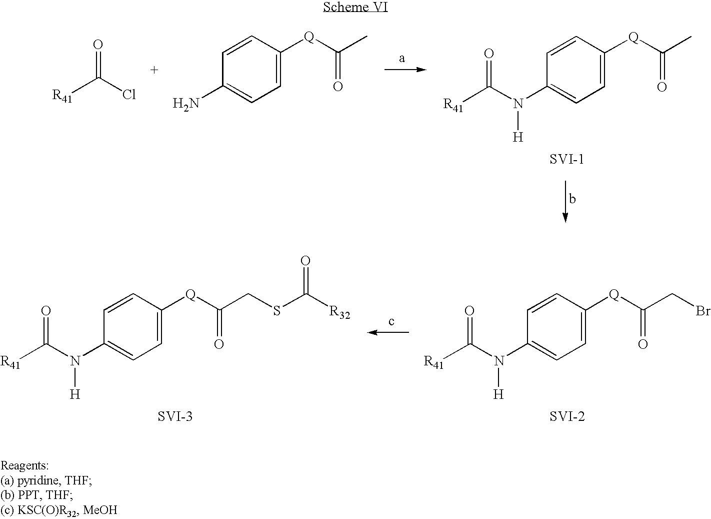

General Procedure for the Synthesis of Reversed Amides:

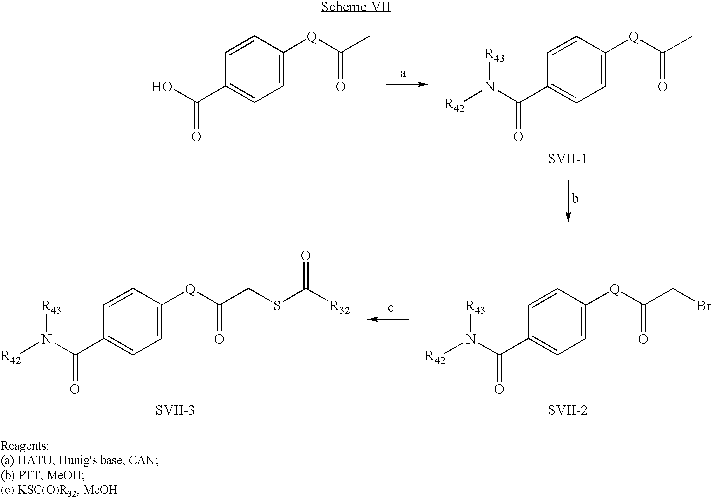

General Procedure for the Synthesis of Tertiary Amines:

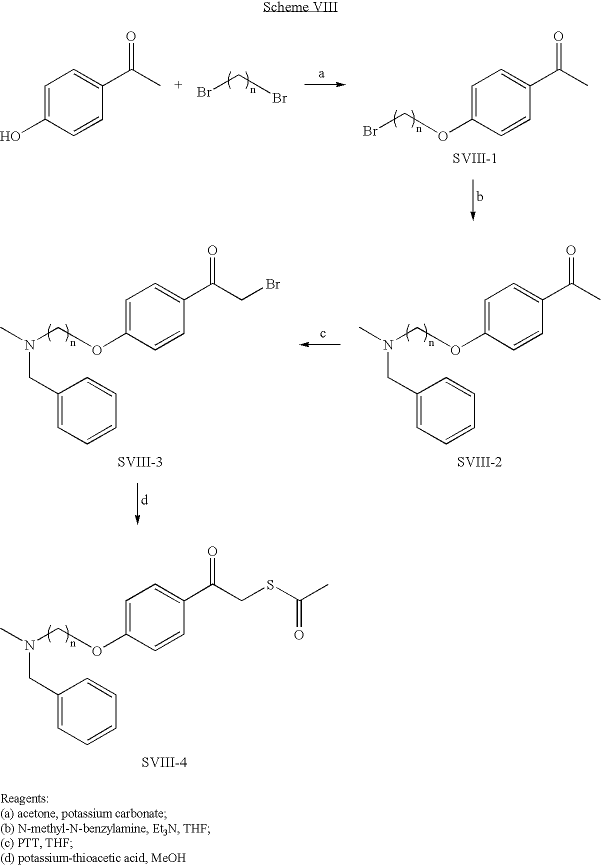

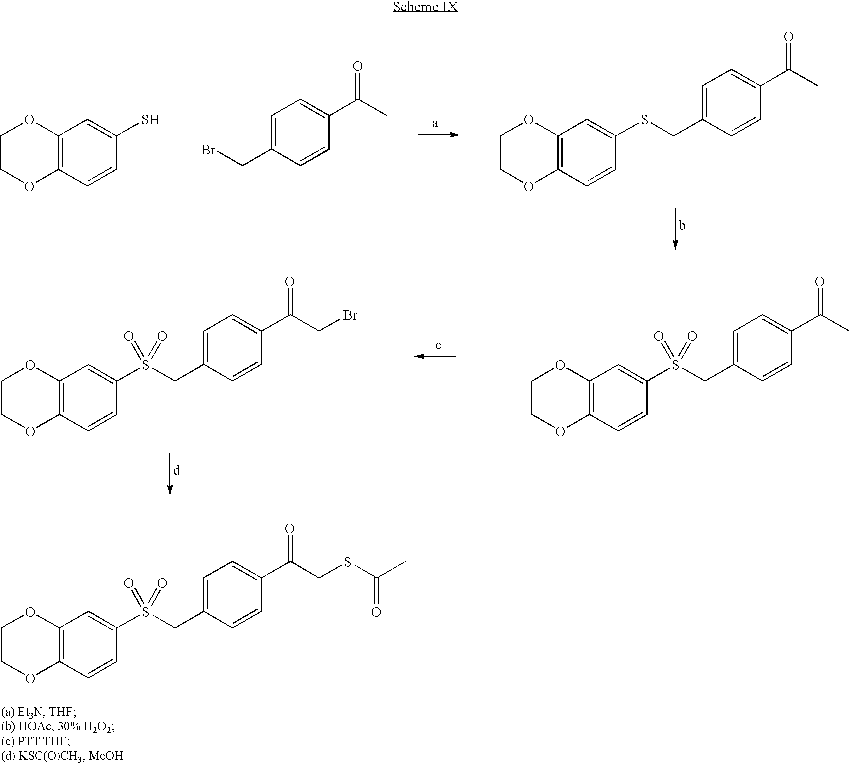

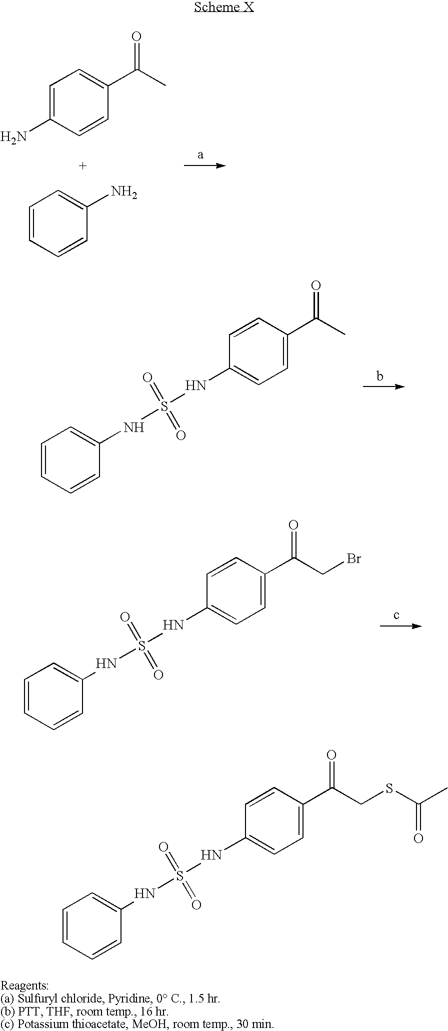

General Procedure for the Synthesis of Mereaptans and Disulfides:

Scheme XI illustrates the general synthesis of disulfide embodiments of the present invention.

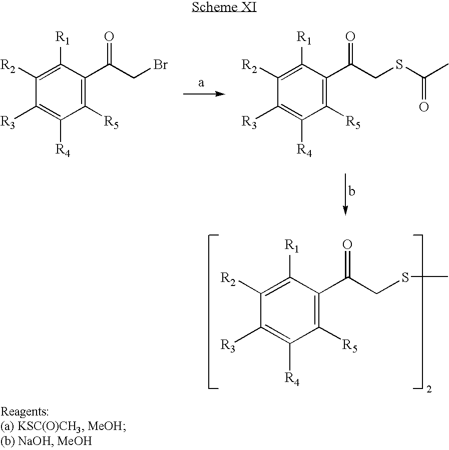

Scheme XII depicts an alternative general scheme for the synthesis of thiol (mercaptan) and disulfide embodiments of the present invention.

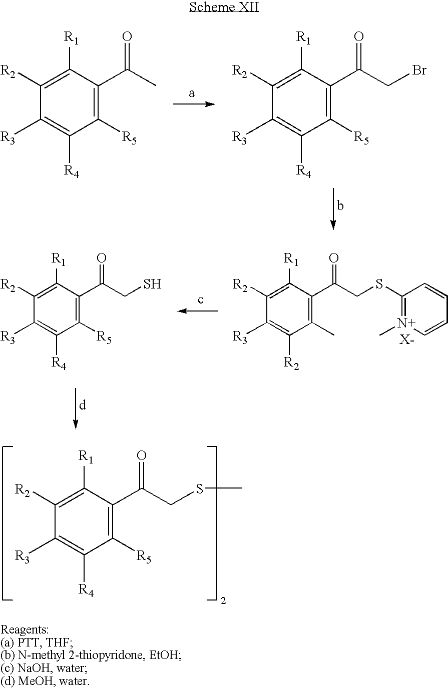

### Example 1

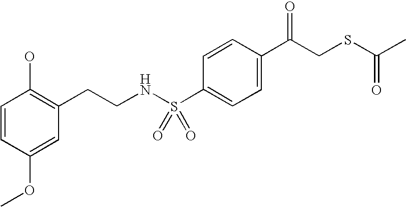

### Thioacetic acid S-(2-{4-12-(2,5-dimethoxy-phenyl)-ethylsulfamoyl]-phenyl}-2-oxo7ethyl)ester

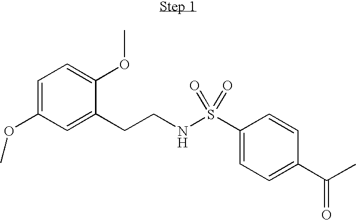

4-Acetyl-N-[2-(2,5-dimethoxy-phenyl)-ethyl]-benzenesulfonamide: To a solution of the amine (0.878 g, 4.84 mmol) in THF (8 mL) was added pyridine (1.174 ml, 14.5 mmol). 4-Acetyl-benzenesulfonyl chloride (1.112 g, 5.05 mmol) was then added as a solid, and the resulting dark solution was stirred for 10 min. Volatiles were removed in vacuo and the resulting residue was suspended in THF. Excess Et₃N was added and the mixture was stirred for several minutes before the solids were filtered. The mother liquor was evaporated to a solid which was recrystalized in ethyl acetate and hexanes to yield the desired compound (1.65 g, 94%). LC-MS (ES+): 364 [MH]⁺ m/e.

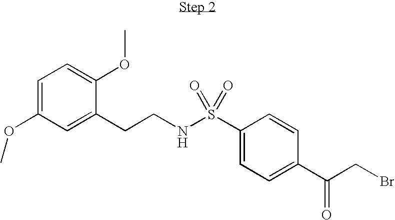

### 4-(2-Bromo-acetyl)-N-[2-(2,5-dimethoxy-phenyl)-ethyl]-benzenesulfonamide

To a solution of the product from step 1 (1.65 g, 4.55 mmol) in THF (9.5 mL) was added phenyltrimethylammonium tribromide (PTT) (1.71 g, 4.55 mmol). The reaction mixture was stirred for 1.5 h and water (5 ml) was added. Volatiles were removed in vacuo and the aqueous mixture was extracted with ethyl acetate. The combined organic solution was dried over over Na₂SO₄ and concentrated in vacuo to afford the desired compound as a white crystalline solid (80%), LC-MS (ES−): 441, 443 m/e.

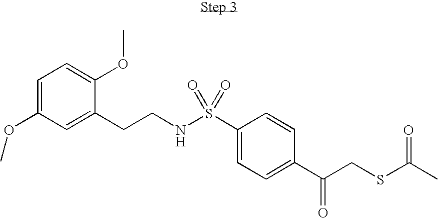

Thioacetic acid S-(2-{4-[2-(2,5-dimethoxy-phenyl)-ethylsulfamoyl]-phenyl}-2-oxo-ethyl)ester: To a solution of the product from step 2 (1.2 g, 2.70 mmol) in methanol (9 mL) was added potassium thioacetate (0.340 g, 2.97 mmol). The reaction solution was stirred at room temperature for ten minutes. Volatiles were removed in vacuo to leaves a tan residue which was dissolved in dichloromethane (4 mL), during which the disulfide of the thioacetic acid was deposited and filtered. The desired thioester could then be recrystallized from dichloromethane/hexanes (0.300 g, 0.686 mmol, 25%). ¹H-NMR (400 MHz, DMSO-d₆): 10.9 (s, 1H), 8.18 (d, 2H) 7.93 (m, 2H), 6.83 (d, 1H), 6.67 (m, 2H), 4.58 (s, 2H), 3.35 (s, 6H), 2.64 (t, 2H), 2.52 (t, 2H), 2.40 (s, 3H); LC-MS (ES+): 438 [MH]⁺ m/e.

### Example 2

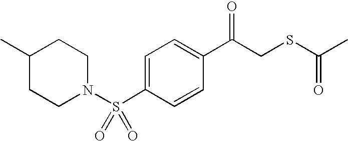

### Thioacetic acid S-{2-[4-(4-methyl-piperidine-1-sulfonyl)-phenyl]-2-oxo-ethyl}ester

The compound, thioacetic acid S-{2-[4-(4-methyl-piperidine-1-sulfonyl)-phenyl]-2-oxo-ethyl}ester, was synthesized according to the procedure described in Example 1. ¹H NMR: (400 MHz, DMSO-d6) δ 8.11 (d, 2H), 7.86 (d, 2H), 4.60 (s, 2H), 3.62 (d, 2H), 2.40 (s, 3H), 2.22 (t, 2H), 1.62 (d, 2H), 1.30 (m, 1H), 1.13 (q, 2H), 0.92 (d, 3H); LC-MS (ES+): 356 [M]⁺ m/e.

### Example 3

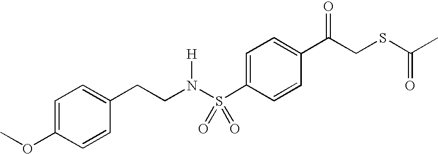

Thioacetic acid S-(2-{4-[2-(4-methoxy-phenyl)-ethylsulfamoyl]-phenyl}-2-oxo-ethyl)ester: The compound, thioacetic acid S-(2-{4-[2-(4-methoxy-phenyl)-ethylsulfamoyl]-phenyl}-2-oxo-ethyl)ester was synthesized according to the procedure described in Example 1. ¹H NMR (400 MHz, DMSO-d6) δ 8.19 (d, 2H), 7.91 (d, 2H), 7.03 (d, 2H), 6.80 (d, 2H), 4.39 (s, 2H), 3.69 (s, 3H), 2.98 (q, 2H), 2.80 (t, 2H), 2.40 (s, 3H); LCMS (ES+): 408 [M]⁺ m/e.

### Example 4

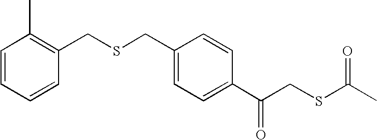

### Thioacetic acid S-{2-[4-(2-methyl-benzylsulfanylmethyl)-phenyl]-2-oxo-ethyl}ester:

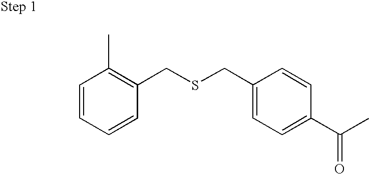

1-[4-(2-Methyl-benzylsulfanylmethyl)-phenyl]-ethanone: To a solution of the thiol (1.5 g, 10.8 mmol) in THF (10 mL) was added Et₃N (4.51 mL, 32 mmol) and the bromo-benzylphenone (2.31 g, 10.8 mmol). The reaction mixture was stirred overnight at room temperature and the white precipitate was removed by filtration. The clear filtrate was concentrated in vacuo. The residue was purified by column chromatography to produce the desired compound as a white crystalline solid (2.08 g, 71%). ¹H NMR (400 MHz, DMSO-d₆) δ 7.93 (d, 2H), 7.46 (d, 2H), 7.14 (m, 4H), 3.78 (s, 2H), 3.65 (s, 2H), 2.57 (s, 3H), 2.24 (s, 3H). LC-MS (ES+): 271 [MH]⁺ m/e.

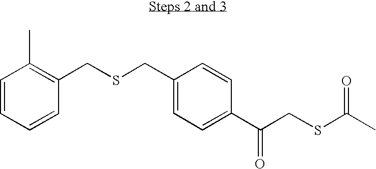

Thioacetic acid S-{2-[4-(2-methyl-benzylsulfanylmethyl)-phenyl]-2-oxo-ethyl}ester: The compound thioacetic acid S-{2-[4-(2-methyl-benzylsulfanylmethyl)-phenyl]-2-oxo-ethyl}ester was synthesized from the product of step 1 according to the procedure described in Example 1, steps 2 and 3. ¹H NMR (400 MHz, CDCl₃) δ 7.96 (d, 2H), 7.42 (d, 2H), 7.15 (m, 4H), 4.39 (s, 2H), 3.68 (s, 2H), 3.60 (s, 2H), 2.41 (s, 3H), 2.30 (s, 3H). LC-MS (ES+): 345 [MH]⁺m/e.

### Example 5

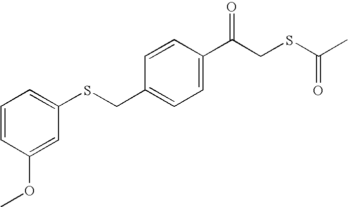

Thioacetic acid S-{2-[4-(3-methoxy-phenylsulfanylmethyl)-phenyl]-2-oxo-ethyl}ester: The compound, thioacetic acid S-{2-[4-(3-methoxy-phenylsulfanylmethyl)-phenyl]-2-oxo-ethyl}ester, was synthesized according to the method described in Example 4. ¹H NMR (400 MHz, DMSO-d₆) δ 7.91 (d, 2H), 7.40 (d, 2H), 7.15 (t, 1H), 6.87 (d, 1H), 6.81 (s, 1H), 6.78 (d, 1H), 4.36 (s, 2H), 4.13 (s, 2H), 3.74 (s, 3H), 2.40 (s, 3H). LC-MS (ES+): 347 [MH]⁺m/e.

### Example 6

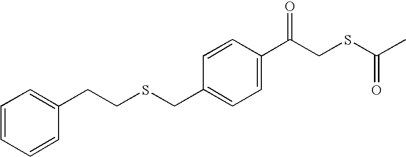

### Thioacetic acid S-[2-oxo-2-(4-phenethylsulfanylmethyl-phenyl)-ethyl]ester

The compound, thioacetic acid S-[2-oxo-2-(4-phenethylsulfanylmethyl-phenyl)-ethyl]ester, was synthesized according to the method described in Example 4.

¹H NMR (400 MHz, DMSO-d₆) δ 7.99 (d, 2H), 7.50 (d, 2H) 7.29 (d, 2H), 7.22 (d, 3H), 4.52 (s, 2H), 3.86 (s, 2H), 2.8 (t, 2H), 2.64 (t, 2H), 2.39 (s, 3H). LC-MS (ES+): 345 [MH]⁺m/e.

### Example 7

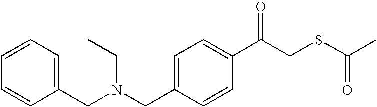

### Thioacetic acid S-(2-{4-[(benzyl-ethyl-amino)-methyl]-phenyl}-2-oxo-ethyl)ester

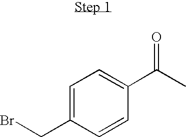

1-(4-Bromomethyl-phenyl)-ethanone: To a solution of 4′-Methyl acetophenone (200 g, 1.49 mol) was in CH₂Cl₂ (1.4 L) was added NBS (265.30 g, 1.49 mol) and AIBN (24.48 g, 0.15 mol). The resulting mixture was stirred under a floodlight (reflux) for 3 h and cooled to room temperature. The crystalline precipitate was removed by filtration and the filtrate was concentrated in vacuo to give a viscous oil which deposited several batches of crystals of the desired product (150 g, 47%) upon standing.

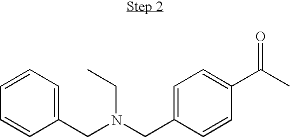

1-{4-[(Benzyl-ethyl-amino)-methyl]-phenyl}-ethanone: To a solution of N-ethyl benzyl amine (137 mg, 1.01 mmol) in THF (3 mL) was added Et₃N (308 mg, 3.04 mmol). 1-(4-Bromomethyl-phenyl)-ethanone from step 1 (216 mg, 1.01 mmol) was then added and the resulting mixture was stirred at room temperature for 1 h. Solids were removed by filtration and the mother liquor was concentrated in vacuo to give an oil which was subjected to radial chromatography. The desired compound was obtained as a clear oil (171 mg, 63%).

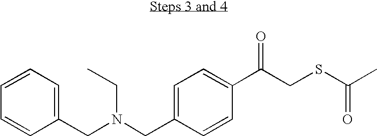

Thioacetic acid S-(2-{4-[(benzyl-ethyl-amino)-methyl]-phenyl}-2-oxo-ethyl)ester: The compound, thioacetic acid S-(2-{4-[(benzyl-ethyl-amino)-methyl]-phenyl}-2-oxo-ethyl)ester, was synthesized from the product of step 2 according to the procedure described in Example 1, steps 2 and 3. ¹H NMR (400 MHz, DMSO-d₆) δ 9.81 (s, 1H), 8.10 (d, 2H), 7.70 (d, 2H), 7.49 (m, 5H), 4.55 (s, 2H), 4.41 (bs, 2H), 4.33 (bs, 2H), 3.00 (q, 2H), 2.58 (s, 3H), 1.28 (t, 3H).

### Example 8

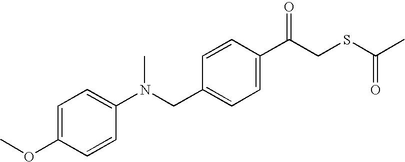

### Thio acetic acid S-[2-(4-{[(4-methoxy-phenyl)-methyl-amino]-methyl}-phenyl)-2-oxo-ethyl]ester

The compound, thioacetic acid S-[2-(4-{[(4-methoxy-phenyl)-methyl-amino]-methyl}-phenyl)-2-oxo-ethyl]ester, was synthesized according to the procedure described in Example 7. ¹H NMR (400 MHz, CDCl₃) δ 7.95 (d, 2H), 7.36 (d, 2H), 7.15 (d, 2H), 6.90 (d, 2H), 4.59 (s, 2H), 4.37 (s, 2H), 3.81 (s, 3H), 3.20 (s, 3H), 2.42 (s, 3H). LC-MS (ES+): 344 [MH]⁺ m/e.

### Example 9

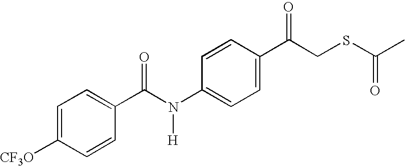

### Thioacetic acid S-{2-oxo-2-[4-(4-trifluoromethoxy-benzoylamino)-phenyl]-ethyl}ester

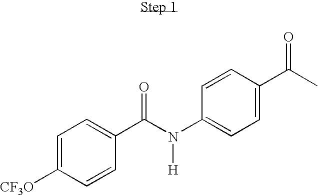

N-(4-Acetyl-phenyl)-4-trifluoromethoxy-benzamide: To a solution of 4′-aminoacetophenone (2.0 g, 14.8 mmol) in anhydrous THF (20 mL) was added 4-(trifluoromethoxy)benzoyl chloride (3.3 g, 14.8 mmol) and pyridine (3.5 g, 44.4 mmol). The reaction mixture was stiffed for 16 h at room temperature. The solid was collected by filtration and triturated in EtOAc (40 mL) to afford the desired product (1.23 g, 26%) as a white solid. LC-MS (ES+): 324 [MH]⁺ m/e.

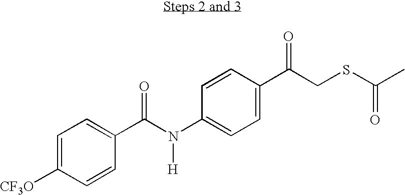

Thioacetic acid S-{2-oxo-2-[4-(4-trifluoromethoxy-benzoylamino)-phenyl]-ethyl)ester: The compound, thioacetic acid S-{2-oxo-2-[4-(4-trifluoromethoxy-benzoylamino)-phenyl]-ethyl)ester, was synthesized from the product of step 1 according to the procedure described in Example 1, steps 2 and 3. ¹H NMR (400 MHz, DMSO-d₆) δ 8.12 (d, 2H), 8.04 (d, 2H), 7.98 (d, 2H), 7.60 (d, 2H), 4.54 (s, 2H), 3.37 (s, 3H). LC-MS (ES+): 398 [MH]⁺ m/e.

### Example 10

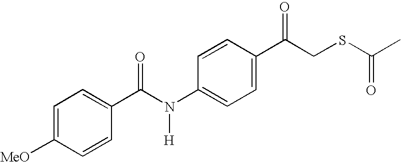

Thioacetic acid S-{2-[4-(4-methoxy-benzoylamino)-phenyl]-2-oxo-ethyl}ester: The compound, thioacetic acid S-{2-[4-(4-methoxy-benzoylamino)-phenyl]-2-oxo-ethyl}ester, was synthesized according to the method described in Example 9. ¹H NMR (400 MHz, CDCl₃) δ 10.43 (s, 1H), 8.02 (d, 2H), 8.00 (d, 2H), 7.99 (d, 2H), 7.10 (d, 2H), 4.51 (s, 2H), 3.83 (s, 2H). LC-MS (ES+): 344 [MH]⁺ m/e.

### Example 11

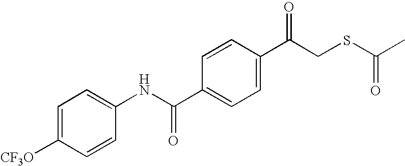

### Thioacetic acid S-{2-oxo-2-[4-(4-trifluoromethoxy-phenylcarbamoyl)-phenyl]-ethyl}ester

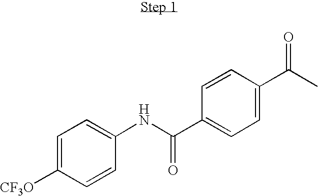

4-Acetyl-N-(4-trifluoromethoxy-phenyl)-benzamide: To a solution of 4-acetylbenzoic acid (3 g, 18.3 mmol) in acetonitrile (75 mL) was added 4-(trifluoromethoxy)aniline, HATU (7.6 g, 20 mmol) and disiisopropylethylamine (5.9 g, 46 mmol). The reaction mixture was stirred for 12 h and the desired product (4.28 g, 13.2 mmol, 72%) was obtained as a white solid.

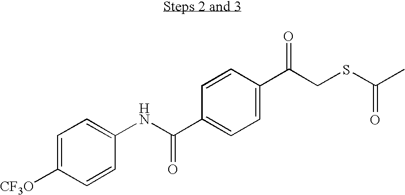

Thioacetic acid S-{2-oxo-2-[4-(4-trifluoromethoxy-phenylcarbamoyl)-phenyl]-ethyl}ester: The compound, thioacetic acid S-{2-oxo-2-[4-(4-trifluoromethoxy-phenylcarbamoyl)-phenyl]-ethyl}ester, was synthesized from the product of step 1 according to the procedure described in Example 1, steps 2 and 3. ¹H NMR (400 MHz, DMSO-d₆) δ 10.62 (s, 1H), 8.19 (d, 2H), 8.10 (d, 2H), 7.92 (d, 2H), 7.40 (d, 2H), 4.60 (s, 2H), 2.60 (s, 3H). LC-MS (ES+): 398 [MH]⁺ m/e.

### Example 12

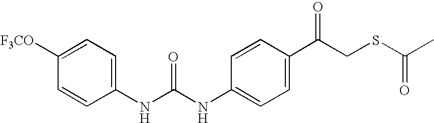

### Thioacetic acid S-(2-oxo-2-{4-13-(4-trifluoromethoxy-phenyl)-ureido]-phenyl}-ethyl)ester

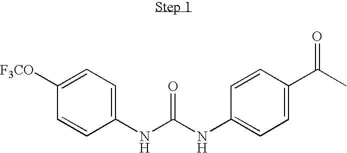

1-(4-Acetyl-phenyl)-3-(4-trifluoromethoxy-phenyl)-urea: To a solution of 4-acetylphenyl isocyanate (1 g, 6.2 mmol) in THF (10 mL) was added triethylamine (1.25 g, 12.4 mmol) and 4-(triflouromethoxy)aniline. The reaction mixture was stirred for 1.5 h. The product was collected by filtration to afford the desired compound (1.17 g, 56%) as a white solid. LC-MS (ES+): 339 [MH]⁺ m/e.

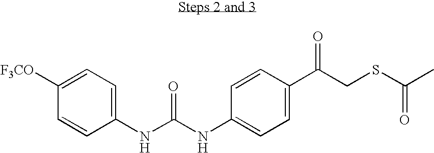

Thioacetic acid S-(2-oxo-2-{4-[3-(4-trifluoromethoxy-phenyl)-ureido]-phenyl}-ethyl)ester: The desired compound, thioacetic acid S-(2-oxo-2-{4-[3-(4-trifluoromethoxy-phenyl)-ureido]-phenyl)-ethyl) ester, was synthesized from the product of step 1 according to the procedure described in Example 1, steps 2 and 3. ¹H NMR (400 MHz, DMSO-d₆) δ 9.12 (s, 1H), 9.05 (s, 1H), 8.00 (d, 2H), 7.62 (d, 2H), 7.60 (d, 2H), 7.32 (d, 2H), 4.47 (s, 2H), 2.40 (s, 3H). LC-MS (ES+): 413 [MH]⁺ m/e.

### Example 13

This example intentionally left blank.

### Example 14

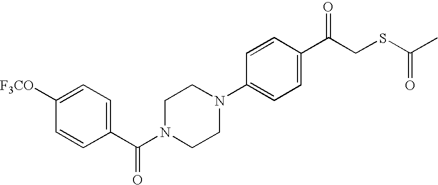

### Thioacetic acid S-(2-oxo-2-{4-[4-(4-trifluoromethoxy-benzoyl)-piperazin-1-yl]-phenyl}-ethyl)ester

To a solution of 4′-piperazino-acetophenone (0.5 g, 2.45 mmol) in anhydrous CH₂Cl₂ (5 mL) was added 4-(trifluoromethoxy)benzenesulfonyl chloride (0.64 g, 2.45 mmol) and triethylamine (0.5 g, 4.9 mmol). The reaction mixture was stirred at room temperature for 3 h. The mixture was concentrated in vacuo and the residue was partitioned between 30 mL of EtOAc and water. The organic layer was washed with 5% sodium bicarbonate and brine. The solution was dried (Na₂SO₄) and concentrated in vacuo to afford the desired product (0.816 g, 68%) as a white solid. ¹H NMR (400 MHz, CDCl₃) δ 7.98 (d, 2H), 7.52(d, 2H), 7.31 (d, 2H), 6.90 (d, 2H), 4.39 (s, 2H), 3.96 (bs, 2B), 3.63 (bs, 2H), 3.42 (bs, 4H), 2.40 (s, 3H). LC-MS (ES+): 467 [MH]⁺m/e.

### Example 15

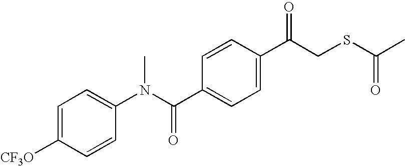

Thioacetic acid S-(2-{4-[methyl-(4-trifluoromethoxy-phenyl)-carbamoyl]-phenyl}-2-oxo-ethyl) ester: The compound thioacetic acid S-(2-{4-[methyl-(4-trifluoromethoxy-phenyl)-carbamoyl]-phenyl}-2-oxo-ethyl)ester was synthesized according to the method described in the preparation of Example 11. ¹H NMR (400 MHz, CDCl₃) δ 7.62 (d, 2H), 7.40 (d, 2H), 7.10 (d, 4H), 4.35 (s, 2H), 4.52 (s, 3H), 2.40 (s, 3H).

### Example 16

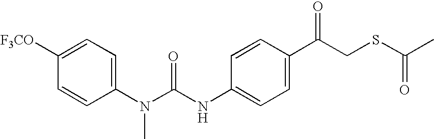

### Thioacetic acid S-(2-{4-[3-methyl-3-(4-trifluoromethoxy-phenyl)-ureido]-phenyl}-2-oxo-ethyl) ester:

The compound, thioacetic acid S-(2-{4-[3-methyl-3-(4-trifluoromethoxy-phenyl)-ureido]-phenyl}-2-oxo-ethyl)ester, was synthesized according to the method described in the preparation of Example 12. ¹H NMR (400 MHz, CDCl₃) δ 8.81 (s, 1H), 7.93 (d, 2H), 7.62 (d, 2H), 7.43 (d, 2H), 7.40 (d, 2H), 4.43 (s, 2H), 3.32 (s, 3H), 2.40 (s, 3H).

### Example 17

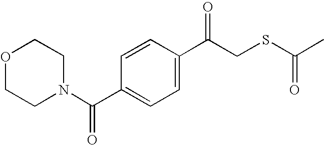

Thioacetic acid S-{2-[4-(morpholine-4-carbonyl)-phenyl]-2-oxo-ethyl}ester: The compound, thioacetic acid S-{2-[4-(morpholine-4-carbonyl)-phenyl]-2-oxo-ethyl}ester, was synthesized according to the method described in the preparation of Example 11. ¹H NMR (400 MHz, CDCl₃) δ 8.08 (d, 2H), 7.54 (d, 2H), 4.40 (s, 2H), 3.81 (bs, 4H), 3.63 (bs, 2H), 3.42 (bs, 2H), 2.40 (s, 3H). LC-MS (ES+): 308 [M]⁺ m/e.

### Example 18

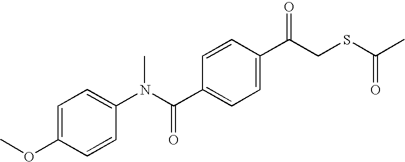

Thioacetic acid S-(2-{4-[(4-methoxy-phenyl)-methyl-carbamoyl]-phenyl}-2-oxo-ethyl)ester: The compound, thioacetic acid S-(2-{4-[(4-methoxy-phenyl)-methyl-carbamoyl]-phenyl}-2-oxo-ethyl)ester, was synthesized according to the method described in the preparation of Example 11. ¹H NMR (400 MHz, CDCl₃) δ 7.80 (d, 2H), 7.40 (d, 2H), 6.99 (d, 2H), 6.78 (d, 2H), 4.38 (s, 2H), 3.79 (s, 3H), 3.48 (s, 3H), 2.40 (s, 3H).

### Example 19

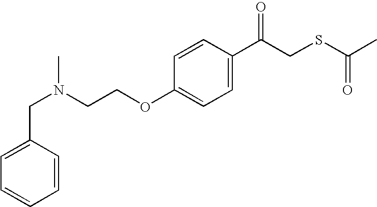

### Thioacetic acid-S-(2-{4-[2-(Benzyl-methyl-amino)-ethoxy]-phenyl}-2-oxo-ethyl)-ester

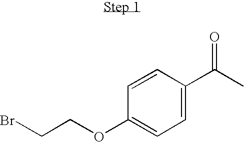

1-[4-(2-Bromo-ethoxy)-phenyl]-ethanone: To a mixture of potassium carbonate (6.09 g, 44.04 mmol) in dry acetone (50 mL) was added 4-hydroxyacetophenone (2.0 g, 14.68 mmol) and the mixture stirred at room temperature for 15 min giving a light yellow solution with white salts at the bottom. To the mixture was added 1,2-dibromoethane (1.29 mL, 14.98 mmol) in acetone (10 mL). The reaction was stirred for 4 h at room temperature, then for 18 h at 40° C. The mixture was filtered through a small plug of celite and the filtrate was concentrated in vacuo to afford the desired compound (2.51 g, 71%) as a white solid which was used directly in the following step. ¹H NMR (400 MHz, CDCl₃) δ 7.98(d, 2H), 7.02(d, 2H), 4,40(t, 2H), 3.70(t, 2H), 2.60(s, 3H). MS: (243.2)

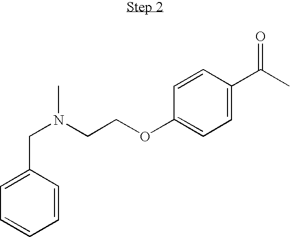

1-{4-[2-(Benzyl-methyl-amino)-ethoxy]-phenyl}-ethanone: To a solution of N-methyl-N-benzylamine (956 μL, 7.41 mmol) in THF was added the product from step 1 (1.8 g, 7.41 mmol) in dry THF. Triethylamine (3 mL, 22 mmol) was added and the reaction stirred at room temperature for 16 h. The solids were removed by filtration and the organic solution was concentrated in vacuo to a yellow oil. The crude material was purified by Semi-prep HPLC to afford (1.03 g, 49%) of pure material. ¹H NMR (400 MHz, CDCl₃) δ 7.99(d, 1H), 7.91(d, 2H), 7.51(d, 2H), 7.41(d, 1H), 6.98(d, 1H), 6.91(d, 2H), 2.88(d, 2H), 2.72(d, 2H), 2.61(s, 3H), 2.58(s, 3H). MS: (284.6)

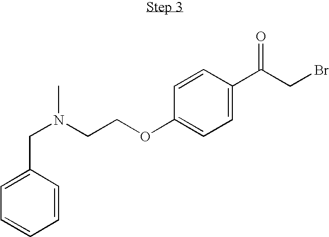

1-{4-[2-(Benzyl-methyl-amino)-ethoxy]-phenyl}-2-bromo-ethanone: To a solution of the product from from step 2 (200 mg, 0.70 mmol) in THF in (20 mL) was added PTT (290 mg, 0.77 mmol). The reaction was stirred at room temperature for 12 h upon which time a light yellow solution with white salts was observed. The reaction was concentrated in vacuo to a light orange solid then partitioned between EtOAc and water. The organic layer was washed twice with water, then brine, dried (Na₂SO₄) and concentrated in vacuo to afford the desired product (250 mg) which was used in the next synthetic step. ¹H NMR (400 MHz, CDCl₃) δ 7.75(d, 2H), 7.14(dd, 2H), 7.07(d, ]H), 7.05(d, 2H), 6.86(d, 2H), 4.56(s, 2H), 4.04(t, 2H), 3.6(s, 2H), 2.80(t, 2H), 2.27(s, 3H); MS (362.8)

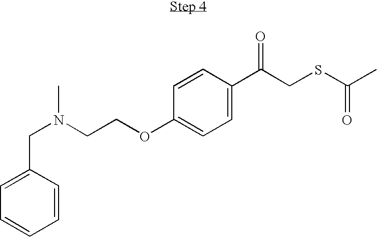

Thioacetic acid-S-(2-{4-[2-(Benzyl-methyl-amino)-ethoxy]-phenyl}-2-oxo-ethyl)-ester: To a solution of the product from step 3 (200 mg, 0.55 mmol) in MeOH (8 mL) was added potassium thioacetate (79 mg, 0.68 mmol). The reaction mixture was stirred at room temperature for 12 h. The mixture was concentrated to a yellow oil. To the residue was added CH₂Cl₂ and solids were removed by filtration. The organic layer was concentrated in vacuo and purified by Semi-prep HPLC to afford the desired product (76 mg, 58%). ¹H NMR (400 MHz, CDCl₃) δ 8.01(d, 1H), 7.91(d, 2H), 7.50(d, 2H), 7.41(d, 1H), 6.99(d, 1H), 6.91(d, 2H), 4.50(s, 2H), 4.39(s, 2H), 2.88(d, 2H), 2.60(s, 3H), 2.44(d, 2H), 2.41(s, 3H). MS: (358.1)

### Example 20

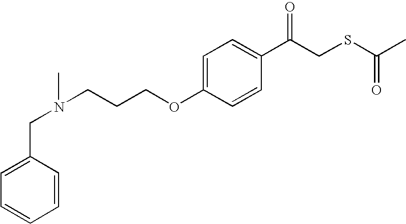

Thioacetic acid-S-(2-{4-[3-(Benzyl-methyl-amino)-propoxy]-phenyl}-2-oxo-ethyl)-ester: The compound, thioacetic acid-S-(2-{4-[3-(Benzyl-methyl-amino)-propoxy]-phenyl}-2-oxo-ethyl)-ester, was synthesized according to the procedure described in the preparation of Example 19. ¹H NMR (400 MHz, CDCl₃) δ 8.00(d, 2H), 7.89(t, 1H), 7.49(d, 4H), 6.90(d, 2H), 4.39(s, 2H), 4.19(m, 2H), 3.41(t, 2H), 2.80(s, 2H), 2.59(t, 2H), 2.43(s, 3H), 2.39(s, 3H). MS: (372.1)

### Example 21

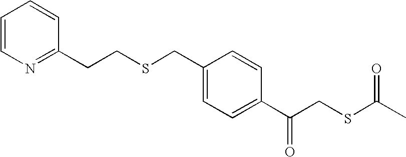

Thioacetic acid S-{2-oxo-2-[4-(2-pyridin-2-yl-ethylsulfanylmethyl)-phenyl]-ethyl}ester: The compound, thioacetic acid S-{2-oxo-2-[4-(2-pyridin-2-yl-ethylsulfanylmethyl)-phenyl]-ethyl}ester, was synthesized according to the procedure described in Example 4. ¹H NMR (400 MHz, CDCl₃) δ 8.54 (d, 1H), 7.91 (m, 2H), 7.60 (t, 1H), 7.39 (d, 2H), 7.15 (m, 2H), 3.80 (s, 2H), 3.10 (t, 2H), 3.0 (t, 2H), 2.37 (s, 3H). LC-MS (ES+): 346 [MH]⁺ m/e.

### Example 22

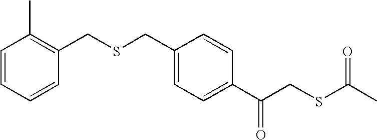

Thioacetic acid S-{2-[4-(2-methyl-benzylsulfanylmethyl)-phenyl]-2-oxo-ethyl}ester: The compound, thioacetic acid S-{2-[4-(2-methyl-benzylsulfanylmethyl)-phenyl]-2-oxo-ethyl}ester, was synthesized according to the procedure described in Example 4. ¹H NMR (400 MHz, CDCl₃) δ 7.96 (d, 2H), 7.42 (d, 2H), 7.15 (m, 4H), 4.39 (s, 2H), 3.68 (s, 2H), 3.60 (s, 2H), 2.41 (s, 3H), 2.30 (s, 3H). LC-MS (ES+): 345 [MH]⁺ m/e.

### Example 23

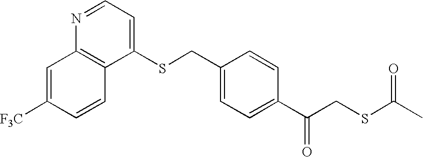

### Thioacetic acid S-{2-oxo-2-[4-(7-trifluoromethyl-quinolin-4-ylsulfanylmethyl)-phenyl]-ethyl}ester

The compound, thioacetic acid S-{2-oxo-2-[4-(7-trifluoromethyl-quinolin-4-ylsulfanylmethyl)-phenyl]-ethyl}ester, was synthesized according to the procedure described in Example 4. ¹H NMR (400 MHz, DMSO-d6) δ 8.82 (m, 1H), 8.28 (m, 2H), 7.95 (d, 2H), 7.88 (d, 1H), 7.66 (m, 3H), 4.65 (s, 2H), 4.47 (s, 2H), 2.35 (s, 3H). LC-MS (ES+): 436 [MH]⁺ m/e.

### Example 24

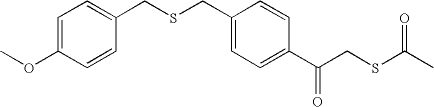

Thioacetic acid S-{2-[4-(4-methoxy-benzylsulfanylmethyl)-phenyl]-2-oxo-ethyl}ester: The compound, thioacetic acid S-{2-[4-(4-methoxy-benzylsulfanylmethyl)-phenyl]-2-oxo-ethyl}ester, was synthesized according to the procedure described in Example 4. ¹H NMR (400 MHz, DMSO-d6) δ 7.94 (d, 2H), 7.39 (d, 2H), 7.17 (d, 2H), 6.85 (d, 2H), 4.39 (s, 2H), 3.80 (s, 3H), 3.61 (s, 2H), 3.55 (s, 2H), 2.41 (s, 3H). LC-MS (ES+): 361 [MH]⁺ m/e.

### Example 25

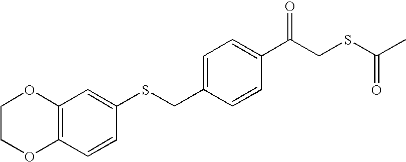

Thioacetic acid S-{2-[4-(2,3-dihydro-benzo[1,4]dioxin-6-ylsulfanylmethyl)-phenyl]-2-oxo-ethyl}ester: The compound, thioacetic acid S-{2-[4-(2,3-dihydro-benzo[1,4]dioxin-6-ylsulfanylmethyl)-phenyl]-2-oxo-ethyl}ester, was synthesized according to the procedure described in Example 4. ¹H NMR (400 MHz, CDCl₃) δ 7.90 (d, 2H), 7.33 (m, 2H), 6.85 (m, 1H), 6.77 (m, 2H), 4.39 (s, 2H), 4.25 (m, 4H), 4.05 (s, 2H), 2.41 (s, 3H). LC-MS (ES+): 375 [MH]⁺ m/e.

### Example 26

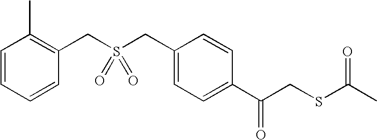

Thioacetic acid S-[2-oxo-2-(4-o-tolylmethanesulfonylmethyl-phenyl)-ethyl]ester: The compound, thioacetic acid S-[2-oxo-2-(4-o-tolylmethanesulfonylmethyl-phenyl)-ethyl]ester, was synthesized according to the procedure described in Example 27. ¹H NMR (400 MHz, DMSO-d6) δ 8.05 (d, 2H), 7.59 (d, 2H), 7.26 (m, 4H), 4.71 (s, 2H), 4.54 (s, 4H), 2.39 (s, 3H), 2.33 (s, 3H). LC-MS (ES+): 377 [MH]⁺ m/e.

### Example 27

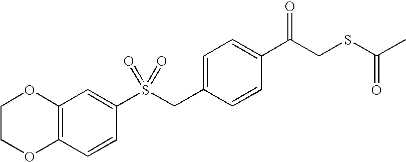

### Thioacetic acid S-{2-[4-(2,3-dihydro-benzo[1,4]dioxine-6-sulfonylmethyl)-phenyl]-2-oxo-ethyl}ester

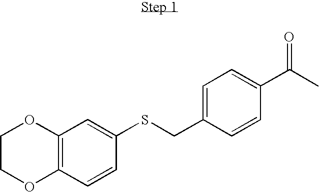

### 1-[4-(2,3-dihydro-benzo[1,4]dioxin-6-ylsulfanylmethyl)-phenyl]-ethanone

To a solution of 3,4-(Ethylenedioxy)thiophenol (0.900g, 5.35 mmol) in THF (10 mL) was added triethylamine (2.23 mL, 0.016 mol). To the reaction mixture was added 1-(4-Bromomethyl-phenyl)-ethanone (1.2 g, 5.62 mmol) as a solid with stirring. The reaction mixture was concentrated in vacuo and the desired thioether (1.12 g, 70% yield) was recrystallized from ethyl acetate and hexanes. LC-MS (ES+): 301 [MH]⁺ m/e.

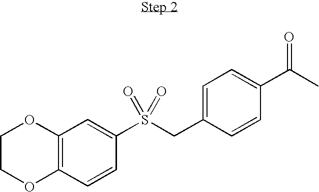

### 1-[4-(2,3-Dihydro-benzo[1,4]dioxine-6-sulfonylmethyl)-phenyl]-ethanone

The product from step 1 (0.474 g, 1.58 mmol) in glacial acetic acid (1.44 ml, 25.28 mmol) was added 30% hydrogen peroxide (0.637 ml, 22.12 mmol). The reaction was heated to 138° C. After 1.5 hours the reaction was removed from heat and allowed to cool to room temperature. Water (15 ml) was then added to the reaction mixture causing the precipitation of a white solid which was filtered and dried (0.394 g, 75%). LC-MS (ES+): 333 [MH]⁺ m/e.

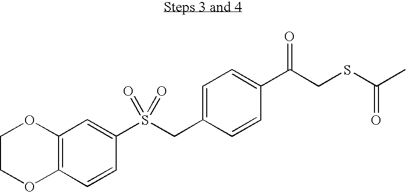

Thioacetic acid S-{2-[4-(2,3-dihydro-benzo[1,4]dioxine-6-sulfonylmethyl)-phenyl]-2-oxo-ethyl}ester: The compound, thioacetic acid S-{2-[4-(2,3-dihydro-benzo[1,4]dioxine-6-sulfonylmethyl)-phenyl]-2-oxo-ethyl)ester, was synthesized from the product of step 2 according to the procedure described in Example 1, steps 2 and 3. ¹H NMR (400 MHz, DMSO-d6) δ 7.91 (d, 2H), 7.27 (d, 2H), 7.20 (d, 1H), 7.08 (q, 1H), 6.90 (d, 1H), 4.37 (s, 2H), 4.33 (m, 4H), 4.28 (m, 2H), 2.41 (s, 3H). LC-MS (ES+): 407 [MH]⁺ m/e.

### Example 28

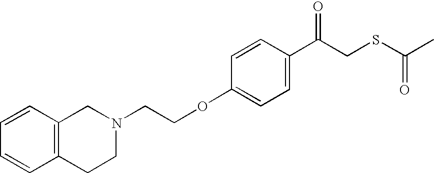

Thioacetic acid S-(2-{4-[2-(3,4-dihydro-1H-isoquinolin-2-yl)-ethoxy]-phenyl}-2-oxo-ethyl)ester: The compound, thioacetic acid S-(2-{4-[2-(3,4-dihydro-1H-isoquinolin-2-yl)-ethoxy]-phenyl}-2-oxo-ethyl)ester, was synthesized according to the procedure described in Example 19. ¹H NMR (400 MHz, CDCl₃) δ 7.98(d, 2H), 7.90(d, 1H), 7.79(dd, 1H), 7.57(d, 1H), 7.41(d, 1H), 6.98(d, 2H), 4.61(t, 2H), 4.57(t, 2H), 4.32(s, 2H), 4.22(t, 2H), 3.71(s, 2H), 3.31(t, 2H), 2.41(s, 3H). MS: (369.41)

### Example 29

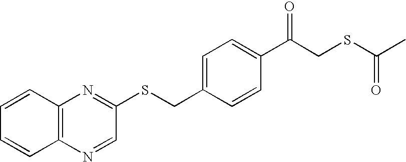

Thioacetic acid S-{2-oxo-2-[4-(quinoxalin-2-ylsulfanylmethyl)-phenyl]-ethyl}ester: The compound, thioacetic acid S-{2-oxo-2-[4-(quinoxalin-2-ylsulfanylmethyl)-phenyl]-ethyl}ester, was synthesized according to the procedure described in Example 4. ¹H NMR (400 MHz, CDCl₃) δ 8.59 (s, 1H), 7.99 (m, 4H), 7.72 (t, 1H), 7.62 (m, 3H), 4.62 (s, 2H), 4.35 (s, 2H), 2.39 (s, 3H). LC-MS (ES+): 3.69 [MH]⁺ m/e.

### Example 30

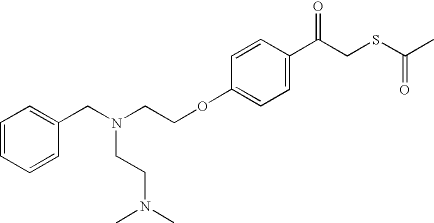

Thioacetic acid S-[2-(4-{2-[benzyl-(2-dimethylamino-ethyl)-amino]-ethoxy}-phenyl)-2-oxo-ethyl]ester: The compound, thioacetic acid S-[2-(4-{2-[benzyl-(2-dimethylamino-ethyl)-amino]-ethoxy)-phenyl)-2-oxo-ethyl]ester, was synthesized according to the procedure described in Example 19. ¹H NMR (400 MHz, CDCl₃) δ 8.01(d, 2H), 7.29(dd, 3H), 6.92(d, 2H), 6.89(d, 2H), 4.40(t, 2H), 4.36(s, 2H), 3.83(t, 2H), 3.65(t, 4H); 3.38(s, 2H), 2.82(s, 6H), 2.41(s, 3H). MS: (414.20)

### Example 31

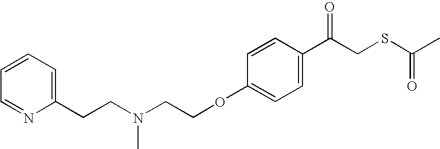

### Thioacetic acid S-[2-(4-{2-[methyl-(2-pyridin-2-yl-ethyl)-amino]-ethoxy}-phenyl)-2-oxo-ethyl]ester

The compound, thioacetic acid S-[2-(4-{2-[methyl-(2-pyridin-2-yl-ethyl)-amino]-ethoxy}-phenyl)-2-oxo-ethyl]ester, was synthesized according to the procedure described in Example 19. ¹H NMR δ (400 MHz, CDCl₃) 8.69(d, 1H), 8.33(dd, 1H), 7.98(d, 2H), 7.96(d, 1H), 7.80(d, 1H), 6.96(d, 2H), 4.43(t, 2H), 4.36(s, 2H), 3.78(t, 2H), 3.70(t, 2H), 3.68(t, 2H), 3.06(s, 3H), 2.40(s, 3H). MS: (372.15)

### Example 32

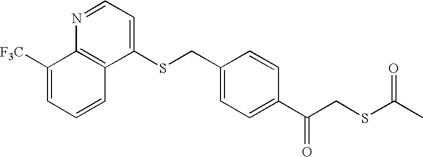

### Thioacetic acid S-{2-oxo-2-[4-(8-trifluoromethyl-quinolin-4-ylsulfanylmethyl)-phenyl]-ethyl}ester

The compound, thioacetic acid S-{2-oxo-2-[4-(8-trifluoromethyl-quinolin-4-ylsulfanylmethyl)-phenyl]-ethyl}ester, was synthesized according to the procedure described in Example 4. ¹H NMR (400 MHz, CDCl₃) δ 8.83 (d, 1H), 8.36 (d, 1H), 8.10 (d, 1H), 7.99 (d, 2H), 7.57 (m, 4H), 4.37 (d, 4H), 2.40 (s, 3H). LC-MS (ES+): 436 [MH]⁺ m/e.

### Example 33

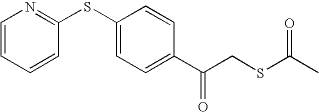

### Thioacetic acid S-{2-oxo-2-[4-(pyridin-2-ylsulfanyl)-phenyl]-ethyl}ester

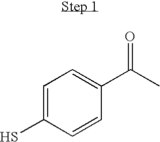

1-(4-Mercapto-phenyl)-ethanone: To a solution of 1-(4-Methylsulfanyl-phenyl)-ethanone (1.0 g, 6.01 mmol) in DMF (8 mL) was added NaSMe (1.0 g, 14.2 mmol). The reaction mixture was heated to 130° C. for 1.5 h. The reaction mixture was cooled to ambient temperature and poured into a mixture of dilute citric acid, ether and ice. The organic phase was separated and the aqueous phase was extracted with ether. The combined organic layers were washed with brine. The organic solution was dried (Na₂SO₄) and concentrated in vacuo to afford the desired product (0.913 g, 100%). LC-MS (ES−): 151 [MH]⁻ m/e.

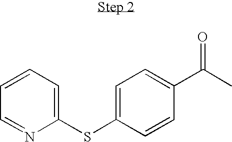

1-[4-(Pyridin-2-ylsulfanyl)-phenyl]-ethanone: To a solution of I-(4-Mercapto-phenyl)-ethanone (1) crude in DMF (5 mL) was added CuCl₂ (0.091 g, 0.60 mmol) and K₂CO₃ (1.180 g, 8.54 mmol). To the reaction mixture was then added 2-bromopyridine (0.594 ml, 6.0 mmol) and the mixture was heated to 100° C. for 6 h. The DMF was removed in vacuo and ethyl acetate (5 mL) was added to the residue. The organic layer was washed with water (2×5 mL). The organic phase was separated, dried (Na₂SO₄) and concentrated in vacua. The residue was purified by column chromatography to yield the desired product (0.56 g, 40%). LC-MS (ES+): 230[MH]⁺ m/e.

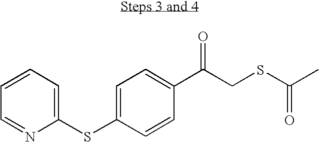

Thioacetic acid S-{2-oxo-2-[4-(pyridin-2-ylsulfanyl)-phenyl]-ethyl}ester. The compound, thioacetic acid S-{2-oxo-2-[4-(pyridin-2-ylsulfanyl)-phenyl]-ethyl}ester, was synthesized from the product of step 2 according to the procedure described in Example 1, steps 2 and 3. ¹H NMR (400 MHz, CDCl₃) δ 8.5 (m, 1H), 7.98 (d, 2H), 7.60 (m, 3H), 7.26 (m, 2H), 4.37 (s, 2H), 2.41 (s, 3H). LC-MS (ES+): 304 [MH]⁺ m/e.

### Example 34

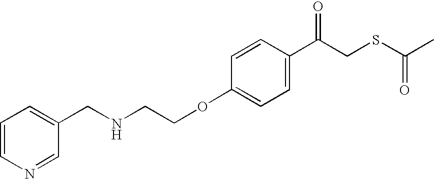

Thioacetic acid S-[2-oxo-2-(4-{2-[(pyridin-3-yl-methyl)-amino]-ethoxy}-phenyl)-ethyl]ester: The compound, thioacetic acid S-[2-oxo-2-(4-{2-[(pyridin-3-yl-methyl)-amino]-ethoxy}-phenyl)-ethyl]ester, was synthesized according to the procedure described in Example 19. ¹H NMR (400 MHz, CDCl₃) δ 8.82(s, 1H), 8.64(d, 1H), 8.31(d, 1H), 7.99(d, 2H), 7.62(dd, 1H), 6.99(d, 2H), 4.43(t, 2H), 4.41(s, 2H), 4.38(s, 2H), 3.51(t, 2H), 2.41(s, 3H). MS: (344.12)

### Example 35

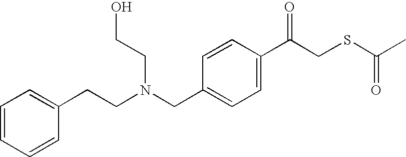

### Thioacetic acid S-[2-(4-{[(2-hydroxy-ethyl)-phenethyl-amino]-methyl}-phenyl)-2-oxo-ethyl]ester

The compound, thioacetic acid S-[2-(4-{[(2-hydroxy-ethyl)-phenethyl-amino]-methyl)-phenyl)-2-oxo-ethyl]ester, was synthesized according to the procedure described in Example 7. ¹H NMR o (400 MHz, CDCl₃) 8.04(d, 2H), 7.61(d, 2H), 7.25(dd, 3H), 7.17(d, 2H), 4.42(s, 2H), 4.38(s, 2H), 3.39(tt, 4H), 3.30(s, 2H), 3.12(t, 2H), 2.41(s, 3H), 2.08(s, 1H). MS: (371.16)

### Example 36

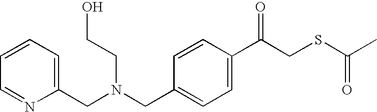

Thioacetic acid S-[2-(4-{(2-hydroxy-ethyl)-pyridin-2-yl-methyl-amino]-methyl}-phenyl)-2-oxo-ethyl]ester. The compound, thioacetic acid S-[2-(4-{[(2-hydroxy-ethyl)-pyridin-2-yl-methyl-amino]-methyl}-phenyl)-2-oxo-ethyl]ester, was synthesized according to the procedure described in Example 7. ¹H NMR (400 MHz, CDCl₃) δ 8.79(d, 1H), 8.01(d, 1H), 7.99(d, 2H), 7.61(d, 2H), 7.59(d, 2H), 4.50(s, 2H), 4.46(s, 2H), 4.38(s, 2H), 3.99(t, 2H), 3.30(t, 2H), 2.41(s, 3H), 2.06(s, 1H). MS: (358.14).

### Example 37

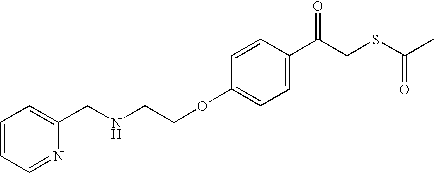

Thioacetic acid S-[2-oxo-2-(4-{2-[(pyridine-2-yl-methyl)-amino]-ethoxy}-phenyl)-ethyl]ester: The compound, thioacetic acid S-[2-oxo-2-(4-{2-[(pyridine-2-yl-methyl)-amino]-ethoxy}-phenyl)-ethyl]ester, was synthesized according to the procedure described in Example 19. ¹H NMR (400 MHz, CDCl₃) δ 8.59(d, 1H), 7.92(d, 2H), 7.80(dd, 1H), 7.44(dd, 1H), 7.32(d, 1H), 6.99(d, 2H), 4.56(t, 2H), 4.46(s, 2H), 4.32(s, 2H), 3:60(t, 2H), 2.41(s, 3H). MS: (344.12).

### Example 38

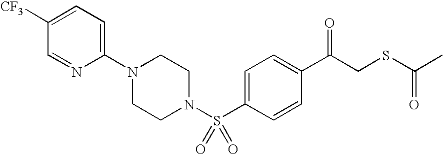

Thioacetic acid S-(2-oxo-2-{4-[4-(5-trifluoromethyl-pyridin-2-yl)-piperazine-1-sulfonyl]phenyl}-ethyl)ester: The compound, thioacetic acid S-(2-oxo-2-{4-[4-(5-trifluoromethyl-pyridin-2-yl)-piperazine-1-sulfonyl]phenyl}-ethyl)ester, was synthesized according to the procedure described in Example 1. ¹H NMR (400 MHz, DMSO-d6) δ 8.19 (s, 1H), 8.11 (d, 2H), 7.90 (d, 2H), 7.80 (d, 1H), 6.92 (d, 1H), 4.58 (s, 2H), 4.72 (t, 4H), 3.02 (t, 4H), 2.38 (s, 3H). LCMS: 489 (M+1)⁺.

### Example 39

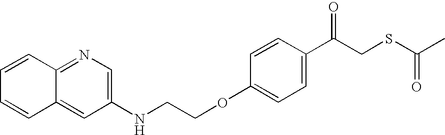

Thioacetic acid S-(2-oxo-2-{4-[2-(quinolin-3-yl-amino)-ethoxy]-phenyl}-ethyl)ester: The compound, thioacetic acid S-(2-oxo-2-{4-[2-(quinolin-3-yl-amino)-ethoxy]-phenyl}-ethyl)ester, was synthesized according to the procedure described in Example 19. ¹H NMR (400 MHz, CDCl₃) δ 9.01(d, 1H), 7.98(s, 1H), 7.82(d, 2H), 7.68(dd, 2H), 7.51(d, 1H), 7.22(s, 1H), 6.84(d, 2H), 4.61(t, 2H), 4.41(s, 2H), 3.58(t, 2H), 2.45(s, 3H). MS: (380.12)

### Example 40

This example intentionally left blank.

### Example 41

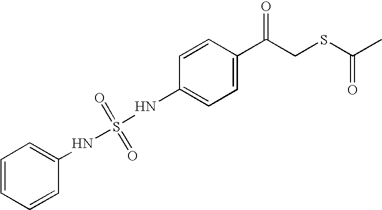

### N-Phenyl-N′-thioacetic acid S-(2-oxo-2-phenyl-ethyl)ester-sulfamide

Step 1

N-Phenyl-N′-(4-acetyl-phenyl)-sulfamide: To a solution of 4-aminoacetophenone (2.0 g, 14.8 mmol) in pyridine (10 mL) was added aniline (1.35 mL, 14.8 mmol). The mixture was stirred at room temperature for 5 min and then cooled to 0° C. in an ice bath for 20 minutes. Sulfuryl chloride (3.9 mL, 44.4 mmol) was added dropwise using caution (large exotherm) over 10 minutes. The reaction was stirred at 0° C. for 30 min and then at room temperature for 1 h. The reaction contents were poured into H₂O (50 mL) and then extracted from ethyl acetate. The organic layers were combined, washed with H₂O, dried (Na₂SO₄) and concentrated in vacuo. The residue was purified by semi-prep HPLC using a mobile phase gradient of 20-60% acetonitrile in H₂O with 0.01% trifluoroacetic acid to give the desired product (846 mg, 20%) as a purple/brown oil. ¹H NMR (400 MHz, CDCl₃) δ 7.98(d, 2H), 7.32(d, 2H), 7.18(d, 2H), 7.04(d, 2H), 6.8(m, 1H), 2.62(s, 3H). MS: (290.1).

### Steps 2 and 3

N-phenyl-N′-thioacetic acid S-(2-oxo-2-phenyl-ethyl)ester-sulfamide: The compound, N-phenyl-N′-thioacetic acid S-(2-oxo-2-phenyl-ethyl)ester-sulfamide, was synthesized from the product of step 1 according to the procedure described in Example 1, steps 2 and 3. ¹H NMR (CDCl₃) δ 7.96(d, 2H), 7,27(m, 2H), 7.16(d, 2H), 7.05(d, 2H), 6.80(dd, 1H), 4.36(s, 2H), 2.42(s, 3H). MS: (365.1).

### Example 42

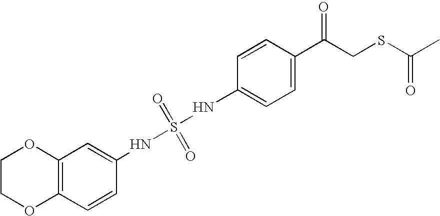

N-(2,3-dihydro-benzo[1,4]dioxane)-N′-thioacetic acid S-(2-oxo-2-phenyl-ethyl)ester-sulfamide: The compound, N-(2,3-dihydro-benzo[1,4]dioxane)-N′-thioacetic acid S-(2-oxo-2-phenyl-ethyl)ester-sulfamide, was synthesized according to the method described in Example 41. ¹H NMR (400 MHz, CDCl₃) δ 7.98(d, 2H), 7.19(d, 2H), 6.73(d, 1H), 6.63(s, 1H), 6.49(d, 1H), 4.37(s, 2H), 4.21(t, 4H), 2.41(s, 3H). MS: (422.1).

### Example 43

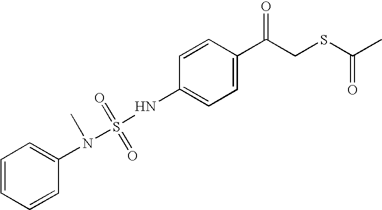

N-methyl-N-phenyl-N′-thioacetic acid S-(2-oxo-2-phenyl-ethyl)ester-sulfamide The compound, N-methyl-N-phenyl-N′-thioacetic acid S-(2-oxo-2-phenyl-ethyl)ester-sulfamide, was synthesized according to the method described in Example 41. ¹H NMR (400 MHz, CDCl₃) δ 8 7.94(d,2H),7.34(d, 2H), 7.22(m, 3H), 7.12(d, 2H), 4.38(s, 2H), 3.32(s, 3H), 2.40(s, 3H). MS: (422.1).

### Example 44

### Thioacetic acid S-{2-oxo-2-[4-(4-trifluoromethoxy-benzylamino)-phenyl]-ethyl}ester

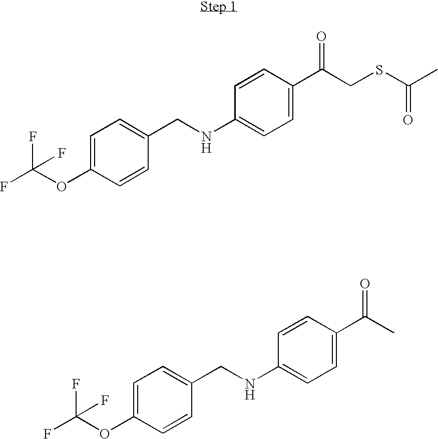

1-[4-(4-Trifluoromethoxy-benzylamino)-phenyl]-ethanone: A mixture of 1-bromomethyl-4-trifluoromethoxy-benzene (500 mg, 1.96 mmol), 1-(4-amino-phenyl)-ethanone (397 mg, 2.94 mmol), and potassium carbonate (433 mg, 3.13 mmol) in acetone (5 mL) was heated to 50° C. for 18 h. The unreacted potassium carbonate was filtered and the filtrate was evaporated to dryness. The resulting crude was purified by flash chromatography (silica gel, 0 to 50% EtOAc:hexane) to afford 333 mg of 1-[4-(4-trifluoromethoxy-benzylamino)-phenyl]-ethanone as a yellow solid. LCMS: 310 (M+1)⁺.

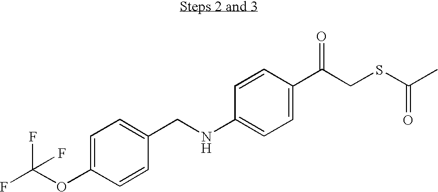

Thioacetic acid S-{2-oxo-2-[4-(4-trifluoromethoxy-benzylamino)-phenyl]-ethyl}ester: The compound, thioacetic acid S-{2-oxo-2-[4-(4-trifluoromethoxy-benzylamino)-phenyl]-ethyl}ester, was synthesized from the product of step 1 according to the procedure described in Example 1, steps 2 and 3. ¹H NMR (400 MHz, DMSO-d₆) δ 7.74 (d, 2H), 7.46 (d, 2H), 7.33 (d, 2H), 6.62 (d, 2H), 4.41 (d, 2H), 4.32 (s, 2H), 2.35 (s, 3H). LCMS: 384 (M+1)⁺.

### Example 45

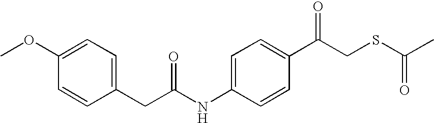

Thioacetic acid S-(2-{4-[2-(4-methoxy-phenyl)-acetylamino]-phenyl}-2-oxo-ethyl)ester: The compound, thioacetic acid S-(2-{4-[2-(4-methoxy-phenyl)-acetylamino]-phenyl}-2-oxo-ethyl)ester, was synthesized according to the method described in Example 9. ¹H NMR (400 MHz, CDCl₃) δ 10.5 (s, 1H), 7.97 (m, 2H), 7.74 (m, 2H), 7.25 (m, 2H), 6.88 (m, 2H), 4.46 (s, 2H), 3.73 (s, 3H), 3.61 (s, 2H), 2.37 (s, 3H). LCMS: 358 (M+1)⁺.

### Example 46

### Thioacetic acid S-{2-oxo-2-[4-(2-phenyl-butyrylamino)-phenyl]-ethyl}ester

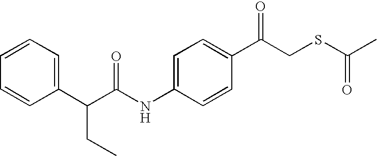

The compound, Thioacetic acid S-{2-oxo-2-[4-(2-phenyl-butyrylamino)-phenyl]-ethyl}ester, was synthesized according to the method described in Example 9. ¹H NMR (400 MHz, CDCl₃) δ 7.87 (m, 2H), 7.69 (s, 1H), 7.55 (m, 2H), 7.31-7.35 (m, 5H), 4.31 (s, 2H), 3.42 (t, 1H), 2.37 (s, 3H), 2.24 (m, 1H), 1.85 (m, 1H), 0.90 (t, 3H). LCMS: 356 (M+1)⁺.

### Example 47

### Thioacetic acid S-{2-oxo-2-[4-(4-phenyl-butyrylamino)-phenyl]-ethyl}ester

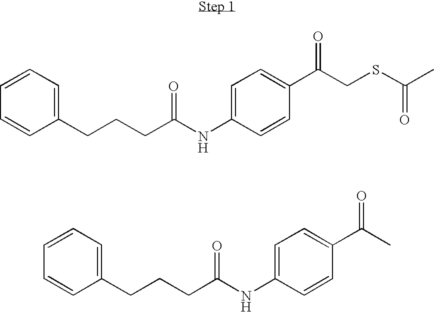

To 4′-Aminoacetophenone (270 mg, 2 mmol), 4-phenylbutyric acid (164 mg, 2 mmol), HOBT (300 mg, 2.2 mmol), HBTU (833 mg, 2.2 mmol) and DIEA (0.4 mL) was added DMF (4 mL) and the resulting reaction mixture was stirred at room temperature for 3 days. The reaction mixture was then poured into EtOAc (100 mL)/HCl (1M, 50 mL) and shaken; the aqueous layer was separated and the organic layer washed with HCL (1M, 50 mL). The organic layer was then washed with NaHCO₃ (sat. aq., 100 mL) dried over Na₂SO₄, filtered and concentrated to a colorless oil which was used without further purification.

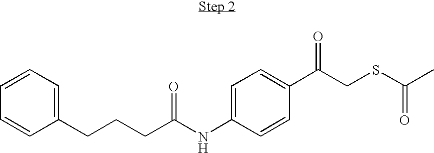

The crude reaction mixture from Step 1 (300 mg, 1.1 mmol) was dissolved in DCM/MeOH/THF (10 mL/1 mL/1 mL) and HBr in acetic acid (33%, 0.5mL) was added followed by PTT (440 mg, 1.2 mmol). The reaction mixture was then stirred at room temperature for 1 hour whereupon it was poured into DCM (100 mL)/pH=7 buffer (1M, 100 mL) and shaken; the organic layer was then dried over Na₂SO₄, filtered and concentrated to give a solid. The crude bromo-ketone was then dissolved in MeOH (6 mL) and sodium thioacetate (140 mg, 1.2 mmol) was added. The reaction mixture was then stirred at room temperature for 24 hours whereupon it was concentrated onto silica gel and purified by flash chromatography (silica gel; EtOAc/Hexane=25/75) to give (Thioacetic acid S-{2-oxo-2-[4-(4-phenyl-butyrylamino)-phenyl]-ethyl}ester) as a white powder (130 mg, 37%). ¹H NMR (400 MHz, CDCl₃) δ 7.96 (m, 2H), 7.61 (m, 2H), 7.19-7.32 (m, 5H), 4.36 (s, 2H), 2.72 (t, 2H), 2.40 (s, 3H), 2.38 (t, 2H), 2.09 (m, 2H). LCMS: 356 (M+1)⁺.

Inhibition Assays

In Vitro HDAC-Inhibition Assay:

This assay measures a compound's ability to inhibit acetyl-lysine deacetylation in vitro and was used as both a primary screening method as well as for IC50 determinations of confirmed inhibitors. The assay is performed in vitro using an HDAC enzyme source (e.g. partially purified nuclear extract or immunopurified HDAC complexes) and a proprietary fluorescent substrate/developer system (HDAC Quantizyme Fluor de Lys Fluorescent Activity Assay, BIOMOL). The assay is run in 1,536-well Greiner white-bottom plates using the following volumes and order of addition:
Step 1: Enzyme (2.5 ul) source added to plate (from refrigerated container)
Step 2: Compounds (50 nl) added with pin transfer device
Step 3: Fluor de Lys (2.5 ul) substrate added, incubate at RT, 30 minutes
Step 4: Developer (5 ul) solution is added (containing TSA), to stop reaction
Step 5: Plate Reader—data collection

The deacetylated fluorophore is excited with 360 nm light and the emitted light (460 nm) is detected on an automated fluorometric plate reader (Aquest, Molecular Devices).

Cellular Histone Hyperacetylation Assays:

These two secondary assays evaluates a compound's ability to inhibit HDAC in cells by measuring cellular histone acetylation levels. The cytoblot facilitates quantitative EC50 information for cellular HDAC inhibition. Transformed cell lines (e.g. HeLa, A549, MCF-7) are cultured under standard media and culture conditions prior to plating.

For Cytoblot:

Cells (approx. 2,500/well) are allowed to adhere 10-24 hours to wells of a 384-well Greiner PS assay plate in media containing 1-5% serum. Cells are treated with appropriate compound and specific concentrations for 0 to 24 hours. Cells are washed once with PBS (60 ul) and then fixed (95% ethanol, 5% acetic acid or 2% PFA) for 1 minute at RT (30 ul). Cells are blocked with 1% BSA for 1 hour and washed and stained with antibody (e.g. anti-Acetylated Histone H3, Upstate Biotechnology), followed by washing and incubation with an appropriate secondary antibody conjugated to HRP or fluorophore. For luminescence assays, signal is generated using Luminol substrate (Santa Cruz Biotechnology) and detected using an Aquest plate reader (Molecular Devices).

For Immunoblot:

Cells (4×10̂5/well) are plated into Corning 6-well dish and allowed to adhere overnight. Cells are treated with compound at appropriate concentration for 12-18 hours at 37 degrees. Cells are washed with PBS on ice. Cells are dislodged with rubber policeman and lysed in buffer containing 25 mM Tris, pH7.6; 150 mM NaCl, 25 mM MgCl2, 1% Tween-20, and nuclei collected by centriguation (7500 g). Nuclei are washed once in 25 mM Tris, pH7.6; 10 mM EDTA, collected by centrifugation (7500 g). Supernatant is removed and histones are extracted using 0.4 M HCl. Samples are centrifuged at 14000 g and supernatants are precipitated in 1 ml cold acetone. The histone pellet is dissolved in water and histones are separated and analyzed by SDS-PAGE Coomassie and immunobloting (anti-acetylated histone antibodies, Upstate Biotechnology) using standard techniques.

Differential Cytotoxicity Assay:

HDAC inhibitors display differential cytotoxicity toward certain transformed cell lines. Cells are cultured according to standard ATCC recommended conditions that are appropriate to each cell type. Compounds were tested for their ability to kill different cell types (normal and transformed) using the ATPlite luminescence ATP detection assay system (Perkin Elmer). Assays are run in either 384-well or 1536-well Greiner PS plates. Cells (30 ul or 5 ul, respectively) are dispensed using either multichannel pipette for 384-well plates, or proprietary Kalypsys bulk liquid dispenser for 1536-well plates. Compounds added using proprietary pin-transfer device (500 nL or 5 nL) and incubated 5 to 30 hours prior to analysis. Luminescence is measured using Aquest plate reader (Molecular Devices).

The activity of some of the compounds of the invention are shown in Table 1 below.

TABLE 1

| | In vitro IC₅₀ | |
|---|---|---|
| Example No. | (μM) | Cellular IC₅₀ (μM) |
| 1 | <1 | <10 |
| 2 | <1 | <50 |
| 3 | <1 | <10 |
| 4 | <1 | N/D |
| 5 | <1 | <10 |
| 6 | <1 | <10 |
| 7 | <1 | <10 |
| 8 | <1 | <10 |
| 9 | <1 | >50 |
| 10 | <1 | <10 |
| 11 | <1 | >50 |
| 12 | <10 | <50 |
| 13 | N/D | N/D |
| 14 | <1 | >50 |
| 15 | <1 | >50 |
| 16 | <1 | <10 |
| 17 | <10 | >50 |
| 18 | <1 | <10 |
| 19 | <1 | <10 |
| 20 | <1 | <10 |
| 21 | <10 | N/D |
| 22 | <1 | N/D |
| 23 | <1 | <1 |
| 24 | <1 | <10 |
| 25 | <1 | <10 |
| 26 | <1 | <10 |
| 27 | <1 | <10 |
| 28 | <1 | <10 |
| 29 | <1 | <10 |
| 30 | <1 | <10 |
| 31 | <1 | <1 |
| 32 | <1 | <1 |
| 33 | <1 | <10 |
| 34 | <10 | <10 |
| 35 | <1 | <10 |
| 36 | <1 | <10 |
| 37 | N/D | N/D |
| 38 | <1 | <10 |
| 39 | <10 | >50 |
| 40 | N/D | N/D |
| 41 | <1 | <1 |
| 42 | <1 | <1 |
| 43 | <1 | <1 |
| 44 | <1 | <10 |
| 45 | <1 | <1 |
| 46 | <1 | <10 |
| 47 | <1 | <10 |

In the table above, N/D indicates that the value was not determined.

What is claimed is:

1. A compound having structural Formula I, 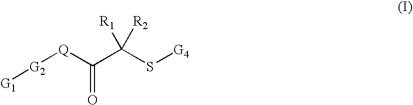
or a pharmaceutically acceptable salt, amide, ester, or prodrug thereof, wherein
G₂ is an optionally substituted phenyl with one or more substituents R₃;
G₁ and G₆ are each independently selected from the group consisting of W and Z;
W is independently selected from the group consisting of
i) an alkoxy of formula —(X₁)ₙ₁—O—(X₂)ₙ₂—X₃, where
each X₁ and each X₂ is each independently selected from the group consisting of optionally substituted lower alkylene, lower alkenylene, lower alkynylene, aryl, and heteroaryl;
X₃ is selected from the group consisting of substituted alkyl, substituted aryl, substituted heteroaryl, optionally substituted heteroalkyl, optionally substituted cycloalkyl; optionally substituted heterocycloalkyl, optionally substituted aralkyl, optionally substituted heteroaralkyl, optionally substituted fused polycyclic aryl and cycloalkyl, optionally substituted fused polycyclic aryl and heterocycloalkyl, optionally substituted linked bi-aryl, optionally substituted linked aryl-heteroaryl, optionally substituted linked heteroaryl-heteroaryl, optionally substituted linked aryl-heterocycloalkyl, an amine of the formula —NX₄X₅, an alkoxy of the the formula —OX₄, and a thioether of the formula —SX₄, where X₄ and X₅ are each independently hydrogen, optionally substituted lower alkyl, optionally substituted aryl, optionally substituted heteroaryl, optionally substituted aralkyl, and optionally substituted heteroaralkyl;
or X₄ and X₅ taken together form an optionally substituted heterocycloalkyl which is optionally fused with optionally substituted aryl or heteroaryl; and
n₁ and n₂ are each independently 0, 1, 2 or 3;
ii) an amide of formula —(X₆)ₙ₃—C(O)—N((X₇)ₙ₄—X₈)X₉ or —(X₆)ₙ₃—N(X₉)C(O)—(X₇)ₙ₄—X₈, where
X₆ and X₇ are each independently selected from the group consisting of optionally substituted lower alkylene, lower alkenylene, lower alkynylene, aryl, and heteroaryl;
X₈ is selected from the group consisting of substituted lower alkyl, optionally substituted aryl, substituted heteroaryl, substituted heteroalkyl, optionally substituted heterocycloalkyl, optionally substituted aralkyl, optionally substituted heteroaralkyl, optionally substituted fused polycyclic aryl and cycloalkyl, optionally substituted fused polycyclic aryl and heterocycloalkyl, optionally substituted linked bi-aryl, optionally substituted linked aryl-heteroaryl, optionally substituted linked heteroaryl-heteroaryl, optionally substituted linked aryl-heterocycloalkyl, an amine of the formula —NX₁₀X₁₁, an alkoxy of the the formula —OX₁₀, and a thioether of the formula —SX₁₀, where X₁₀ and X₁₁ are each independently hydrogen, optionally substituted lower alkyl, optionally substituted aryl, optionally substituted heteroaryl, optionally substituted aralkyl, and optionally substituted heteroaralkyl, provided that X₁₀ is neither H nor unsubstituted alkyl; or, if X₉ is not H, X₈ may additionally be selected from the group consisting of lower alkyl, aryl, heteroaryl, and heteroalkyl;
X₉ is selected from the group consisting of hydrogen, optionally substituted lower alkyl, optionally substituted heteroalkyl, optionally substituted aryl, optionally substituted heteroaryl, optionally substituted heterocycloalkyl, hydroxyl, and optionally substituted alkoxy;
or X₈ and X₉ taken together form an optionally substituted heterocycloalkyl; and
n₃ and n₄ are independently 0, 1, 2, or 3;
iii) an amino of formula —(X₁₂)ₙ₅—N((X₁₃)ₙ₆—X₁₄)X₁₅, where
X₁₂ and X₁₃ are each independently selected from the group consisting of lower alkylene, lower alkenylene, lower alkynylene, aryl, and heteroaryl;
X₁₄ is selected from the group consisting of substituted lower alkyl, optionally substituted aryl, substituted heteroaryl, substituted heteroalkyl, optionally substituted heterocycloalkyl, optionally substituted aralkyl, optionally substituted heteroaralkyl, optionally substituted fused polycyclic aryl and cycloalkyl, optionally substituted fused polycyclic aryl and heterocycloalkyl, optionally substituted linked bi-aryl, optionally substituted linked aryl-heteroaryl, optionally substituted linked heteroaryl-heteroaryl, optionally substituted linked aryl-heterocycloalkyl, an amine of the formula —NX₁₆X₁₇, an alkoxy of the the formula —OX₁₆, and a thioether of the formula —SX₁₆, where X₁₆ and X₁₇ are each independently hydrogen, optionally substituted lower alkyl, optionally substituted aryl, optionally substituted heteroaryl, optionally substituted aralkyl, and optionally substituted heteroaralkyl;
X₁₅ is selected from the group consisting of hydrogen, optionally substituted lower alkyl, optionally substituted heteroalkyl, optionally substituted aryl, optionally substituted heteroaryl, optionally substituted heterocycloalkyl, hydroxyl, and optionally substituted alkoxy;
or n₆ is 0 and X₁₄ and X₁₅, taken together with the nitrogen to which they are attached, form a substituted five-membered or six-membered heteroaromatic or heterocyclic ring;
with the proviso that when X₁₄ and X₁₅ are taken together with the nitrogen to which they are attached to form a six-membered heterocyclic ring that contains an endocyclic oxygen, then it is not N-morpholino; and
each n₅ and each n₆ is each independently 0, 1, 2, or 3;
iv) a thioether or thiol of formula —(X₁₈)ₙ₇—S—(X₁₉)ₙ₈—X₂₀, or the higher oxide forms —(X₁₈)ₙ₇—S(O)—(X₁₉)ₙ₈—X₂₀ and —(X₁₈)ₙ₇—SO₂—(X₁₉))ₙ₈—X₂₀, wherein
X₁₈ and X₁₉ are each independently selected from the group consisting of lower alkylene, lower alkenylene, lower alkynylene, aryl, and heteroaryl;
X₂₀ is selected from the group consisting of substituted lower alkyl, substituted aryl, substituted heteroaryl, optionally substituted heteroalkyl, optionally substituted heterocycloalkyl, optionally substituted aralkyl, optionally substituted heteroaralkyl, optionally substituted fused polycyclic aryl and cycloalkyl, optionally substituted fused polycyclic aryl and heterocycloalkyl, optionally substituted linked bi-aryl, optionally substituted linked aryl-heteroaryl, optionally substituted linked heteroaryl-heteroaryl, optionally substituted linked aryl-heterocycloalkyl, an alkoxy of the the formula —OX₂₁, and a thioether of the formula —SX₂₁, where X₂₁ and X₂₂ are each independently hydrogen, optionally substituted lower alkyl, optionally substituted aryl, optionally substituted heteroaryl, optionally substituted aralkyl, and optionally substituted heteroaralkyl; and
n₇ and n₈ are each independently 0, 1, 2, or 3; further wherein if n8 is 0 and G₁=W, then X₂₀ excludes perfluoroalkyl; further wherein, if G₁=Z and R₂₀=unsubstituted alkyl or unsubstituted aralkyl then X₂O excludes perfluoroalkyl;
v) a moiety of the structure —(X₂₃)ₙ₉—N(X₂₅)C(O)—V—(X₂₄)ₙ₁₀—X₂₆ or —(X₂₃)ₙ₉—V—C(O)N((X₂₄)ₙ₁₀—X₂₅)X₂₆, wherein
V is independently selected from O and S;
X₂₃ and X₂₄ is each independently selected from the group consisting of lower alkylene, lower alkenylene, lower alkynylene, aryl, and heteroaryl;
X₂₅ and X₂₆ is each independently selected from the group consisting of hydrogen, optionally substituted lower alkyl, optionally substituted heteroalkyl, optionally substituted aryl, optionally substituted heteroaryl, optionally substituted heterocycloalkyl, optionally substituted aralkyl, optionally substituted heteroaralkyl, optionally substituted fused polycyclic aryl and cycloalkyl, optionally substituted fused polycyclic aryl and heterocycloalkyl, optionally substituted linked bi-aryl, optionally substituted linked aryl-heteroaryl, optionally substituted linked heteroaryl-heteroaryl, optionally substituted linked aryl-heterocycloalkyl, an amine of the formula —NX₂₇X₂₈, an alkoxy of the the formula —OX₂₇, and a thioether of the formula —SX₂₇, where each X₂₇ and each X₂₈ are each independently hydrogen, optionally substituted lower alkyl, optionally substituted aryl, optionally substituted heteroaryl, optionally substituted aralkyl, and optionally substituted heteroaralkyl;
or n₁₀ is 0 and X₂₅ and X₂₆, taken together with the nitrogen to which they are attached, form an optionally substituted five-membered or six-membered heteroaromatic or heteroaliphatic ring; and
n₉ and n10 are each independently 0, 1, 2, or 3; and
vi) a moiety of the structure —X₂₉—X₃₀, wherein
each X₂₉ is independently selected from the group consisting of optionally substituted C₁-C₁₀ alkylene, optionally substituted C₁-C₁₀ alkenylene, and optionally substituted C₁-C₁₀ alkynylene;
each X₃₀ is independently selected from the group consisting of optionally substituted aryl, optionally substituted heteroaryl, optionally substituted heterocycloalkyl, optionally substituted fused polycyclic aryl and cycloalkyl, optionally substituted fused polycyclic aryl and heterocycloalkyl, optionally substituted linked bi-aryl, optionally substituted linked aryl-heteroaryl, optionally substituted linked heteroaryl-heteroaryl, optionally substituted linked aryl-heterocycloalkyl; further wherein if G₁=Z, then X₃₀ is substituted.
Z is selected from the group consisting of
i) an N-sulfonamido of structure 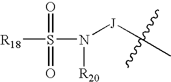
wherein R₁₈ is selected from the group consisting of —(X₃₁)ₙ₁₁—X₃₂, and —NX₃₅X₃₆—, wherein each X₃₁ is independently selected from the group consisting of optionally substituted lower alkylene, lower alkenylene, and lower alkynylene;
X₃₅ is selected from the group consisting of hydrogen, optionally substituted lower alkyl, and optionally substituted lower heteroalkyl;
X₃₆ is selected from the group consisting of optionally substituted aryl, optionally substituted heteroayl, and optionally substituted fused polycyclic aryl and cycloalkyl;
J is —(CH₂)ₖ—, wherein k is 0-3;
each n₁₁ is independently 1, 2, or 3;
each X₃₂ is independently selected from the group consisting of substituted and monocyclic aryl, heteroaryl, cycloalkyl, and heterocycloalkyl;
and wherein R₂₀ is hydrogen, optionally substituted lower alkyl, optionally substituted lower aralkyl, optionally substituted aryl, optionally substituted heteroalkyl, and optionally substituted heteroaralkyl; and
ii) an S-sulfonamido of formula 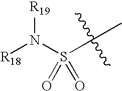
wherein R₁₈ is independently selected from the group consisting of —(X₃₃)ₙ₁₂—X₃₄, wherein X₃₃ is independently selected from the group consisting of optionally substituted lower alkylene, lower alkenylene, and lower alkynylene;
n₁₂ is 1, 2, or 3; and
X₃₄ is a optionally substituted monocyclic phenyl, where the substituents cannot be taken in together to form a ring fused with the phenyl moiety;
wherein R₁₉ is hydrogen, optionally substituted lower alkyl, optionally substituted lower aralkyl, optionally substituted aryl; optionally substituted heteroalkyl, and optionally substituted heteroaralkyl;
or R₁₈ taken together with R₁₉ and the nitrogen to which they are attached forms an optionally substituted heterocycloalkyl;
R₁ and R₂ are each independently selected from the group consisting of hydrogen, optionally substituted lower alkyl, and halogen, or taken together form optionally substituted cycloalkyl;
R₃ is selected from the group consisting of hydrogen, optionally substituted lower alkyl, optionally substituted aryl, optionally substituted heteroaryl, optionally substituted heteroalkyl, halogen, optionally substituted amino, and hydroxyl;
Q is selected from the group consisting of a bond, —(CH₂)ₘ—, —CH₂)ₘNR₂₁—, —CH₂)ₘ(CO)—, —(CH₂)ₘNR₂₁(CO)—, —(CH₂)ₘNR₂₁(CO)— and —(CH₂)ₘC(O)NR₂₁—, wherein m is 0-7, optionally substituted lower alkylene, optionally substituted lower alkynylene, optionally substituted lower heteroalkyl, and optionally substituted lower heteroalkynylene, wherein if Q is not symmetric, Q may be attached in either order;
R₂₁ is selected from the group of hydrogen, alkenyl, and alkyl, wherein alkyl is C₁ to C₈;
G₄ is selected from the group consisting of acyl, aryl, alkyl, heteroaryl, and Z, wherein Z has the structural formula (II) 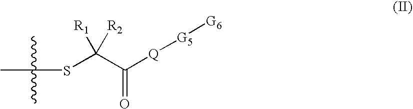
G₅ is selected from the group consisting of monocyclic aryl, polycyclic aryl, monocyclic heteroaryl, and polycyclic heteroaryl; alternatively, G₄ is selected from the group consisting of optionally substituted alkylthio and optionally substituted arylthio to form a disulfide with the alkylthio or arylthio substituents;
With the proviso that the said compound is not the following:

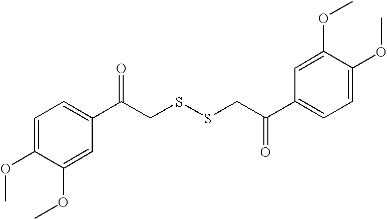

2. The compound of claim 1 wherein Q is a bond.

3. The compound of claim 2 wherein G₁ is W and W is the alkoxy of formula —(X₁)ₙ₁—O—(X₂)ₙ₂—X₃.

4. The compound of claim 3 wherein X₃ is an amine of the formula —NX₄X₅, where X₄ and X₅ are each independently hydrogen, optionally substituted lower alkyl, optionally substituted aryl, optionally substituted heteroaryl, optionally substituted aralkyl, and optionally substituted heteroaralkyl.

5. The compound of claim 2 wherein G₁ is W and W is the amide of formula —(X₆)ₙ₃—C(O)—N((X₇)ₙ₄—X₈)X₉ or —(X₆)ₙ₃—N(X₉)C(O)—(X₇)ₙ₄—X₈.

6. The compound of claim 5 wherein
X₈ is optionally substituted phenyl; and
X₉ is selected from the group consisting of hydrogen, optionally substituted lower alkyl and optionally substituted heteroalkyl.

7. The compound of claim 2 wherein G₁ is W and W is the amino of formula —(X₁₂)ₙ₅—N((X₁₃)ₙ₆—X₁₄)X₁₅.

8. The compound of claim 7 wherein
X₁₄ is selected from the group consisting of substituted lower alkyl, substituted aryl, substituted heteroaryl, substituted heteroalkyl, optionally substituted heterocycloalkyl, optionally substituted aralkyl, optionally substituted heteroaralkyl, optionally substituted fused polycyclic aryl and heterocycloalkyl, optionally substituted linked bi-aryl, optionally substituted linked aryl-heteroaryl, optionally substituted linked heteroaryl-heteroaryl, optionally substituted linked aryl-heterocycloalkyl; and
X₁₅ is selected from the group consisting of hydrogen and optionally substituted lower alkyl.

9. The compound of claim 2 wherein G₁ is W and W is the thioether or thiol of formula —(X₁₈)ₙ₇—S—(X₁₉)ₙ₈—X₂₀.

10. The compound of claim 9 wherein X₁₈ and X₁₉ is alkylene.

11. The compound of claim 10 wherein n7 is 1.

12. The compound of claim 2 wherein G₁ is Z and Z is the N-sulfonamido.

13. The compound of claim 12 wherein R₁₈ is —NX₃₅X₃₆—.

14. The compound of claim 1 wherein R₂₀ and R₂₁ are joined to form an optionally substituted heterocycle.

15. The compound of claim 1 wherein said compound is selected from the group consisting of Examples 1-47.

16. A pharmaceutical composition comprising the compound of claim 1 together with at least one pharmaceutically acceptable carrier, diluent or excipient.

17. The compound of claim 1 wherein the compound or pharmaceutically acceptable salt, amide, ester or prodrug thereof is capable of inhibiting the catalytic activity of histone deacetylase (HDAC).

18. A method of treating a HDAC related disease in a patient in need thereof by administering a compound of claim 1 to said patient.

19. The method of claim 18, wherein said disease is a hyper-proliferative condition of the human or animal body.

20. The method of claim 19 wherein the hyper-proliferative condition is selected from the group consisting of cancer of oral cavity and pharynx, cancer of the digestive system, cancer of the respiratory system, cancer of bones and joints, cancer of soft tissue, skin cancer, breast cancer, cancer of the genital system, cancer of the urinary system, cancer of eye and orbit, cancer of brain and other nervous system, cancer of the endocrine system, cancer of lymphoma, cancer of multiple myeloma and leukemia.

21. The method of claim 20 wherein said hyper-proliferative condition is selected from the group consisting of tongue cancer, mouth cancer, pharynx cancer, other oral cavity cancer, esophagus cancer, stomach cancer, small intestine cancer, colon cancer, rectum cancer, anus cancer, anal canal cancer, anorectum cancer, liver cancer, intrahepatic bile duct cancer, gallbladder and other biliary organs cancer, pancreas cancer, other digestive organs cancer, larynx cancer, lung and bronchus cancer, other respiratory organs cancer, heart cancer, melanoma-skin cancer, basal cancer, squamous cancer, other non-epithelial skin cancer, uterine cervix cancer, uterine corpus cancer, ovary cancer, vulva cancer, vagina and other genital cancer, prostate cancer, testis cancer, penis and other genital cancer, urinary bladder cancer, kidney and renal pelvis cancer, ureter and other urinary organs cancer, thyroid cancer, other endocrine cancer, Hodgkin's disease cancer, non-Hodgkin's lymphoma cancer, acute lumphocytic leukemia, chronica lymphocytic leukemia, acute myeloid leukemia, chronic myeloid leukemia,other leukemias and myeloproliferative disorders such as polycythemia vera, myelofibrosis and essential thrombocythenia.

22. The method of claim 18, wherein said disease is selected from the group consisting of a neurological disorder, and polyglutamine-repeat disorders.

23. The method of claim 22, where the polyglutamine-repeat disorder is selected from the group consisting of Huntington's disease, Spinocerebellar ataxia 1 (SCA 1), Machado-Joseph disease (MJD)/Spinocerebella ataxia 3 (SCA 3), Kennedy disease/Spinal and bulbar muscular atrophy (SBMA) and Dentatorubral pallidolusyian atrophy (DRPLA).

24. The method of claim 19, wherein said disease is an anemia or thalassemia.

25. The method of claim 24, wherein the thalassemia is Sickle Cell Disease.

26. The method of claim 18, wherein said disease is an inflammatory condition.

27. The method of claim 26, wherein the inflammatory condition is selected from the group consisting of Rheumatoid Arthritis (RA), Inflammatory Bowel Disease (IBD), ulcerative colitis and psoriasis.

28. The method of claim 18, wherein said disease is an autoimmune disease.

29. The method of claim 28, wherein the autoimmune disease is selected from the group consisting of Systemic Lupus Erythromatosus (SLE) and Multiple Sclerosis (MS).

30. The method of claim 18, wherein said disease is a cardiovascular condition.

31. The method of claim 30, wherein the cardiovascular condition is selected from the group consisting of cardiac hypertrophy and heart failure.

32. The compound of claim 1 for use in the manufacture of a medicament for the prevention or treatment of a disease or condition ameliorated by the modulation of histone deacetylase (HDAC).
